(19) United States
(12) Patent Application Publication
(10) Pub. No.: US 2026/0348925 A1
(43) Pub. Date: Nov. 5, 2026

(54) DETERMINING HPLC METHOD PARAMETERS USING MACHINE LEARNING

(71) Applicant: Hoffmann-La Roche Inc., Little Falls, NJ (US)

(72) Inventors: Ryan Paul BURWOOD, Stein (CH); Pascal Manuel ZIMMERLI, Muttenz (CH)

(73) Assignee: Hoffmann-La Roche Inc., Little Falls, NJ (US)

(21) Appl. No.: 19/124,484

(22) PCT Filed: Oct. 26, 2023
(86) PCT No.: PCT/EP2023/079867

§ 371 Date: Apr. 25, 2025

(30) Foreign Application Priority Data
Oct. 28, 2022 (EP) 22204521.3

(51) Int. Cl. G01N 30/86; G16C 20/70

(52) U.S. Cl. CPC G01N 30/8662; G01N 30/8693; G16C 20/70

(57) ABSTRACT

Computer-implemented methods for predicting HPLC retention time for one or more compounds using machine learning models are disclosed. In particular, the use of machine learning models for identifying a set of HPLC method parameters suitable for separating two or more compounds in a composition is disclosed. Related systems and products are also described.

### FIELD OF THE DISCLOSURE

The present invention relates to computer-implemented methods for predicting HPLC retention time for one or more compounds using machine learning models. In particular, the present invention relates to the use of machine learning models for identifying a set of HPLC method parameters suitable for separating two or more compounds in a composition, and to related systems and devices.

### BACKGROUND

High-Performance Liquid Chromatography (HPLC) is the most commonly used analytical technique in the development of new drugs. From drug discovery and development to commercial manufacturing, HPLC is used for purity assays, stability studies, identification of degradation and side products, genotoxic limit tests, or preparative separations (Dong & Guillarme, 2013; Snyder et al., 2009).

In the development process of an HPLC method, in order to achieve maximum separation between the compounds, a wide range of parameters such as the choice of mobile phase (eluents), pH of the eluents, gradient program, flow rate, column oven temperature, and the choice of stationary phase (column) need to be optimised. Currently, a time-consuming and costly one-factor-at-a-time ‘trial-and-error’ approach is often applied to find the best settings.

It is therefore desirable to provide methods which address the above mentioned technical challenges.

### SUMMARY OF THE DISCLOSURE

Broadly, the present invention provides machine learning models for predicting HPLC retention time for one or more compounds, and for identifying a set of HPLC method parameters suitable for separating two or more compounds in a composition.

Accordingly, a first aspect provides a method of predicting a HPLC retention time for one or more compounds, the method comprising: (a) obtaining the values of one or more structural and/or physicochemical properties of said compounds, and one or more HPLC method parameters; and (b) using a machine learning model to predict a retention time for each of said compounds when subjected to HPLC using the one or more HPLC method parameters, wherein the machine learning model has been trained using a training dataset comprising molecular structural properties and/or physicochemical properties for one or more compounds, and for each compound, one or more sets of chromatographic data comprising a retention time for the respective compound and associated HPLC method parameters.

The present inventors have identified that it was possible to train a machine learning model to predict chromatographic data including the retention time for respective compounds when separated using associated HPLC method parameters provided as input to the machine learning model, with high prediction accuracy. The inventors further recognised that these highly accurate predictions that are HPLC-method could be used to identify HPLC method parameters suitable for separating two or more compounds in a composition.

Thus, also described herein according to a second aspect is a method of identifying a set of HPLC method parameters suitable for separating two or more compounds in a composition comprising said compounds, the method comprising: (a) performing the method of the first aspect for a plurality of sets of HPLC method parameters; (b) calculating one or more separation performance metrics using the results of step (a); and (c) identifying one or more set(s) of HPLC method parameters by applying one or more criteria on said separation performance metrics.

According to a third aspect there is provided a method for providing a tool for predicting a HPLC retention time for one or more compounds, the method comprising: (a) obtaining a training dataset comprising the values of one or more structural and/or physicochemical properties for one or more compounds, and for each compound, one or more sets of chromatographic data comprising a retention time for the respective compound and associated HPLC method parameters; and (b) training a machine learning model to predict a retention time for a compound when subjected to HPLC using the one or more HPLC method parameters, using as input the values of the one or more structural and/or physicochemical properties for the compound, and values of said HPLC method parameters.

### BRIEF DESCRIPTION OF THE FIGURES

FIG. 1 shows a schematic representation of an HPLC System.

FIG. 2 depicts a flow chart diagram of an example method of predicting a HPLC retention time (A) and identifying a set of HPLC method parameters suitable for separating two or more compounds (B). Steps illustrated in boxes with dashed outlines are optional.

FIG. 3 shows the gradient of SAM-0200368 with the time points marked as tp_1, tp_2 and tp_3.

FIG. 4 is a star schema representing the consolidated data.

FIG. 5 shows definitions and basic operations of a Genetic Algorithm according to an implementation of the methods of the disclosure. In the figure, Flow is flow rate, Temp is temperature, pH, Tp_1 is timepoint 1, Tp_2 is timepoint 2, Tp_3 is timepoint 3, Ep_1 is elution power 1, Ep_2 is elution power 2, Ep_3 is elution power 3, Length is column length, inner_diameter is the column inner diameter, Particle size is the column particle size, Pore_size is the column pore size, H is hydrophobicity, S is steric interaction, A is hydrogen-bond acidity, B is hydrogen-bond basicity, c is ion-exchange capacity.

FIG. 6 shows basic operations of a Genetic Algorithm according to an implementation of the methods of the disclosure.

FIG. 7 shows prediction error plots of the evaluated machine learning models prior to feature selection using random train-test-split according to an implementation of the methods of the disclosure.

FIG. 8 shows comparison of the two splitting methods. Left: random train-test-split (80/20), right: unified train-test-slit (80/20). Y_train and y_test relate to the training and test data, respectively. Using the “unified” sampling approach, an 80/20 ratio between train and test samples was maintained over the entire retention time range.

FIG. 9 shows the top 20 of the most important features of the XGB model using unified train-test-split. The numbers are the F core values also illustrated by the length of the bar.

FIG. 10 shows evaluation of the hyper-parameters for the XGB model after performing unified train-test-split and feature selection, using different learning rates. All models reached good performance. Using a learning_rate of 0.1 and 0.05, the model reached good performance with only a few hundred estimators. The plotted score is the mean test score of the 10-fold CV.

FIG. 11 shows prediction error and residual plot of the final XGB model. The residual plot shows random error over the entire RT range indicating a good fit of the model. The model performs best between 2 min and 20 min, likely due to the number of observations in this range.

FIG. 12 shows prediction error and residual plot using molecular fingerprints and XGB. The histograms show the distribution of the test set as well as the distribution of the predicted values.

FIG. 13 shows comparison of KMeans clustering with 30 and 8 clusters using both ion-exchange parameters (c). PCA was applied before clustering. The “x” denotes the cluster centers.

FIG. 14 shows comparison of different eps values for the clustering of analytical columns using DBSCAN and c_28.

FIG. 15 shows comparison of measured and predicted RT using the SAM method parameters and the XGB model specifically trained for the optimizer. The numbering of the peaks is defined by the RT from Empower data (i.e. measured RT, red peaks).

FIG. 16 shows comparison of the predicted and measured results for the molecules in an example elution (labelled SAM-0114188) using the method parameters from the optimiser.

FIG. 17 shows comparison of the predicted and measured results for the molecules in an example elution (labelled SAM-0113392) using the recommended method parameters by the optimiser.

FIG. 18 shows a screenshot of a user interface for accessing the methods of the disclosure.

### DETAILED DESCRIPTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Further, as used in the following, the terms “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention can, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

A compound as described herein may be a small molecule (e.g. a small molecule inhibitor, activator, cofactor, etc.) or a large molecule (e.g. a biologic, therapeutic protein or peptide such as an antibody or compound derived therefrom, a nucleic acid, etc.). A compound may be an organic compound. A compound may be a pharmaceutically active agent (also referred to as a drug or therapeutic agent), or a degradation product thereof.

The systems and methods described herein may be implemented in a computer system, including or in addition to the structural components and user interactions described. As used herein, the term “computer system” includes the hardware, software and data storage devices for embodying a system or carrying out a method according to the above described embodiments. For example, a computer system may comprise a processing unit such as a central processing unit (CPU) and/or graphics processing unit (GPU), input means, output means and data storage, which may be embodied as one or more connected computing devices. Preferably the computer system has a display or comprises a computing device that has a display to provide a visual output display. The data storage may comprise RAM, disk drives or other computer readable media. The computer system may include a plurality of computing devices connected by a network and able to communicate with each other over that network. It is explicitly envisaged that computer system may consist of or comprise a cloud computer.

The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

High-Performance Liquid Chromatography (HPLC, aka. “high-pressure liquid chromatography”) is the most commonly used analytical technique in the development of new drugs. The strength of HPLC lies in its almost universal applicability as only a few samples cannot be measured using this technique. Through the coupling of HPLC with other techniques such as mass spectrometry (LC-MS), HPLC gained even more importance. From drug discovery and development to commercial manufacturing, HPLC is used for purity assays, stability studies, identification of degradation and side products, genotoxic limit tests, or preparative separations (Dong & Guillarme, 2013; Snyder et al., 2009). In the development process of an HPLC method, a wide range of parameters needs to be set. To achieve maximum separation performance between analytes, several parameters such as the mobile phase (eluents), pH of the eluents, gradient program, flow rate, column oven temperature, and stationary phase (column) must be selected. Currently, determining optimal parameters entail a great deal of manual optimization, involving several trial and error steps, which is time-consuming and leads to increased costs, even though the guidelines available in the field help shorten this procedure to some extent.

In the context of the present invention, the term HPLC is also meant to encompass ultra-high performance liquid chromatography, and HPLC performed on its own or a part of an analytics or separation process, such as e.g. as part of LC-MS.

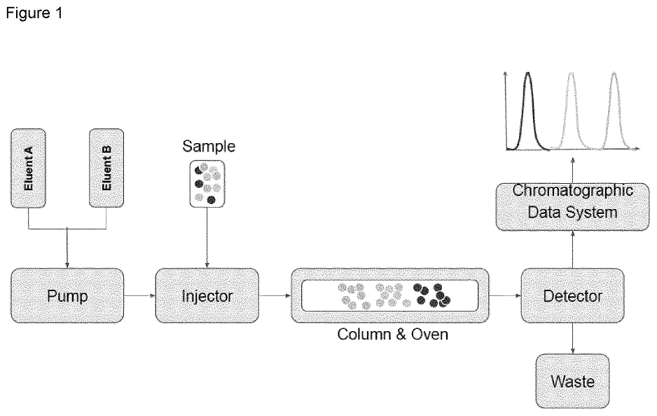

FIG. 1 shows a schematic representation of an HPLC System. After the injection of a liquid sample using the auto-sampler, the eluent flow from the pump carries the sample through the HPLC system. Once the sample enters the column, it is separated into its different components. The separation mode is mainly determined by the column. There are several types of HPLC, such as reversed-phase (RPLC), normal-phase (NP-LC), hydrophilic interaction (HILIC), ion-pair (IPC), size-exclusion (SEC), and affinity chromatography. The methods described herein are applicable to all types of HPLC. Currently, RP-LC is the most commonly used separation mode in the development process of new small molecule drugs, especially for water-soluble samples (Dong, 2006). In embodiments, the HPLC may be RP-LC. In RP-LC, the stationary phase (referred to as the column) is non-polar. The column is a stainless steel tube filled with spherical particles made of porous, modified silica. The most common modification in RP-LC is C-18. These C-18 groups are attached to the surface of each particle providing the column with its non-polar character. The mobile phase consists of a polar mixture of an organic solvent (common examples include acetonitrile or methanol) and water. Different migration (“speed of travel”) of the sample molecules (referred to as analyte) causes the separation in the column. The migration of an analyte is determined by an equilibrium process between molecules of the same analyte present in the mobile phase and in the stationary phase at any time. The stronger an analyte binds to the stationary phase, the stronger its retention by the stationary phase and the higher its retention time (RT). In addition to the chemical structure of the analyte, the mobile and stationary phase, and the temperature influences the migration rate. The sample solvent does not bind to the column due to its polarity and leaves the column first. After being separated on the column, the different molecules leave the column and enter a detector. The most common type is the ultraviolet absorption detector (LC-UV). A UV detector measures the fraction of light that is transmitted through the flow cell containing the sample as a function of time. The relation between the fraction of the transmitted light and the concentration of an analyte in a sample can be described using Beer-Lambert-Law. The output of an LC-UV experiment is a chromatogram, where the absorbance is plotted against the retention time (RT).

As the first aspect, the present invention provides a method of predicting a HPLC retention time for one or more compounds, the method comprising: (a) obtaining the values of one or more structural and/or physicochemical properties of said compounds, and one or more HPLC method parameters; and (b) using a machine learning model to predict a retention time for each of said compounds when subjected to HPLC using the one or more HPLC method parameters, wherein the machine learning model has been trained using a training dataset comprising molecular structural properties and/or physicochemical properties for one or more compounds, and for each compound, one or more sets of chromatographic data comprising a retention time for the respective compound and associated HPLC method parameters.

The methods of the present aspect may have any of the features described in relation to any other aspect.

The step of “obtaining” can comprise calculating the properties, receiving them from a user interface, or from a computing device or database.

The HPLC method parameters can comprise parameters related to the column (e.g. dimensions, particle size), the eluent (e.g. pH of the eluents), or the chromatography procedure (e.g. flow rate).

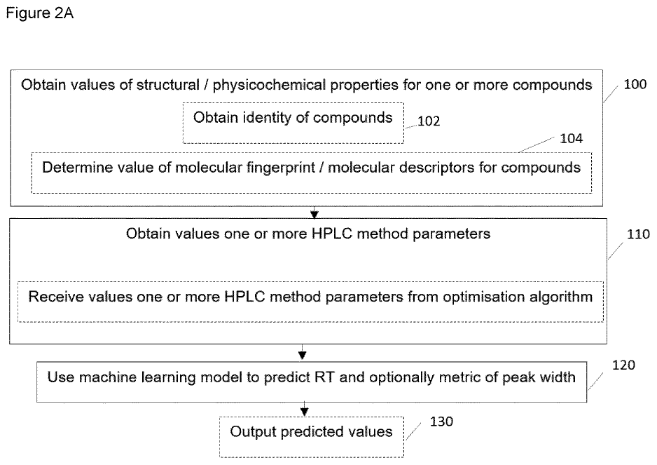

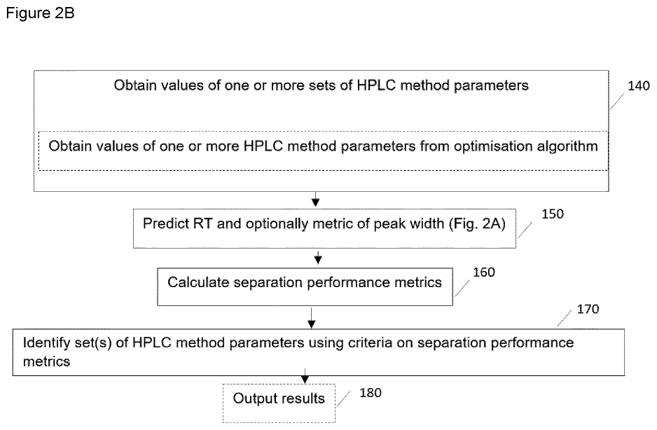

A schematic presentation is provided in FIG. 2A. At step 100, values of one or more structural and/or physicochemical properties for one or more compounds are obtained. This may comprise obtaining the identity of the compounds, for example from a user, at optional step 102. This may comprise optional step 104 of determining the value of the structural/physicochemical properties. Alternatively, these may be received from a user, computing device or database. The properties may comprise a molecular fingerprint and/or one or more molecular descriptors. In embodiments, the structural molecular descriptors and/or molecular fingerprints are 2D molecular descriptors or molecular fingerprints. In embodiments, the molecular descriptors are selected from a group consisting of ABCIndex, AcidBase, AdjacencyMatrix, Aromatic, AtomCount, Autocorrelation, BCUT, BalabanJ, BaryszMatrix, BertzCT, BondCount, CarbonTypes, Chi, Constitutional, DetourMatrix, DistanceMatrix, EState, EccentricConnectivityIndex, ExtendedTopochemicalAtom, FragmentComplexity, Framework, HydrogenBond, InformationContent, KappaShapeIndex, Lipinski, McGowanVolume, MoeType, MolecularDistanceEdge, Molecularld, PathCount, Polarizability, RingCount, RotatableBond, SLogP, TopoPSA, TopologicalCharge, TopologicalIndex, VdwVolumeABC, VertexAdjacencyInformation, WalkCount, Weight, WienerIndex, and ZagrebIndex. In embodiments, said parameters are calculated by Mordred package. In embodiments, the molecular descriptors are selected from a group consisting of SLogP, ATSC5v, ATSC8d, ATSC3Z, ABC, VSA_EState4, ATSC5dv, and ATSC6i.

At step 110, the values of one or more HPLC method parameters are obtained. These may be received from a user, computing device or database. For example, these may be received from an optimisation algorithm executed on a processor. In embodiments, sets of HPLC method parameters are generated by genetic algorithm. In embodiments, the genetic algorithm is run until one or more stopping criteria apply, optionally wherein the stopping criteria are selected from: i) a predetermined number of generations has been reached, and ii) the difference between the separation performance metrics associated with one or more sets of HPLC method parameters of a current iteration and the separation performance metrics associated with one or more sets of HPLC method parameters of a previous iteration is below a threshold. In embodiments, the genetic algorithm is initialised with a set of HPLC method parameters selected randomly, wherein the genetic algorithm is initialised with a set of HPLC method parameters selected from a predetermined set or range for each HPLC method parameter, and/or wherein the genetic algorithm is initialised with a set of HPLC method parameters provided by a user. In embodiments, one or more sets of HPLC method parameters are selected (e.g. by a genetic algorithm) from respective predetermined sets and/or ranges.

At step 120, the machine learning model is used to predict retention time for each of compounds when subjected to HPLC using the one or more HPLC method parameters. Optionally, the machine learning model further predicts a metric indicative of peak width, and the one or more sets of chromatographic data in the training dataset further comprise a metric indicative of the width of the peak for the respective compound.

At step 130, the predicted values are presented to a user, e.g. through a user interface, to a computing device or database.

In an embodiment, the machine learning model further predicts a metric indicative of peak width. In such embodiments, the machine learning model is trained using one or more sets of chromatographic data that further comprise a metric indicative of the width of the peak for the respective compound.

In an embodiment, the one or more compounds are individually selected from: a pharmaceutically active agent, and a degradation product thereof.

In an embodiment, the HPLC method parameters are selected from a group consisting of: HPLC type, flow rate, temperature, pH, column length, column inner diameter, column particle size, column pore size, column steric interaction, column hydrogen bond acidity, column hydrogen bond basicity, column ion exchange capacity (c_28), column ion exchange capacity (c_70), and one or more metrics defining the elution phase gradient. In an embodiment, the HPLC method parameters comprise one or more of the parameters selected from a group consisting of: column length, column inner diameter, column particle size, column pore size, column steric interaction, column hydrogen bond acidity, column hydrogen bond basicity, column ion exchange capacity (c_28), column ion exchange capacity (c_70), and one or more metrics defining the elution phase gradient, and optionally one or more parameters selected from a group consisting of: HPLC type, flow rate, temperature, pH. In an embodiment, the HPLC method parameters comprise HPLC type, flow rate, temperature, pH, and one or more of the parameters selected from a group consisting of: column length, column inner diameter, column particle size, column pore size, column steric interaction, column hydrogen bond acidity, column hydrogen bond basicity, column ion exchange capacity (c_28), column ion exchange capacity (c_70), and one or more metrics defining the elution phase gradient. In an embodiment, the HPLC method parameters comprise one or more of the parameters selected from a group consisting of: column length, column inner diameter, column particle size, column pore size, column steric interaction, column hydrogen bond acidity, column hydrogen bond basicity, column ion exchange capacity (c_28), column ion exchange capacity (c_70), and optionally one or more parameters selected from a group consisting of: HPLC type, flow rate, temperature, pH, and one or more metrics defining the elution phase gradient. In an embodiment, the HPLC method parameters comprise i) HPLC type, flow rate, temperature, one or more metrics defining the elution phase gradient, pH, and ii) one or more of the parameters selected from a group consisting of: column length, column inner diameter, column particle size, column pore size, column steric interaction, column hydrogen bond acidity, column hydrogen bond basicity, column ion exchange capacity (c_28), column ion exchange capacity (c_70).

In embodiments, the HPLC type can be e.g. normal phase (NP) chromatography, reverse phase (RP) chromatography, size exclusion chromatography, ion-exchange chromatography, hydrophilic interaction chromatography (HILIC), and affinity chromatography. In embodiments, the HPLC type is RP or NP chromatography. Specifically, the HPLC type may be RP chromatography.

In an embodiment, the one or more metrics defining the elution phase gradient comprise:
(i) the elution power of each of a plurality of mobile phases; and/or
(ii) metrics defining the time-dependent change in the proportion of a plurality of mobile phases during elution, optionally wherein the one or more metrics defining the time-dependent change in the proportion of a plurality of mobile phase during elution comprise the value of one or more time points corresponding to changes in the rate of change of the proportion of the mobile phases, and/or the value of the rate of change of the proportion of the mobile phases at one or more time points during the elution, and/or metrics describing a function representing the change in proportion of the mobile phases in one or more of (e.g. each of) a plurality of regions in the gradient; and/or
(iii) the mobile phase elution powers at two or more (e.g. three) different time points, optionally wherein the mobile phase comprises a plurality of mobile phases and the elution power at a time point is obtained as a sum of the elution powers of each of the plurality of mobile phases weighted by the respective proportion of each mobile phase.

In an embodiment, the one or more physicochemical properties comprise one or more molecular descriptors and/or wherein the one or more molecular structural properties comprise a molecular fingerprint and/or one or more structural molecular descriptor. In embodiments, the one or more physicochemical properties and/or molecular structural properties are calculated by PaDEL-Descriptor, BlueDesc, ChemoPy, PyDPI, Rcpi, Cinfony, or Dragon software, preferably by Mordred package. Any package available in the art for calculating physicochemical properties and/or molecular structural properties of molecules, for example starting from molecular formulae, 2D or3D structures, may be used.

In an embodiment, the structural molecular descriptors and/or molecular fingerprints are 2D molecular descriptors or molecular fingerprints.

In an embodiment, the molecular descriptors are selected from a group consisting of ABCIndex, AcidBase, AdjacencyMatrix, Aromatic, AtomCount, Autocorrelation, BCUT, BalabanJ, BaryszMatrix, BertzCT, BondCount, CarbonTypes, Chi, Constitutional, DetourMatrix, DistanceMatrix, EState, EccentricConnectivityIndex, ExtendedTopochemicalAtom, FragmentComplexity, Framework, HydrogenBond, InformationContent, KappaShapeIndex, Lipinski, McGowanVolume, MoeType, MolecularDistanceEdge, Molecularld, PathCount, Polarizability, RingCount, RotatableBond, SLogP, TopoPSA, TopologicalCharge, TopologicalIndex, VdwVolumeABC, VertexAdjacencyInformation, WalkCount, Weight, WienerIndex, and ZagrebIndex. In embodiments, said parameters are calculated by Mordred package.

In an embodiment, the one or more structural and/or physicochemical properties comprise molecular descriptors selected from a group consisting of SLogP, ATSC5v, ATSC8d, ATSC3Z, ABC, VSA_EState4, ATSC5dv, and ATSC6i. In embodiments, said parameters are calculated by Mordred package.

In an embodiment, the elution phase gradient (in the training data or in the HPLC method for which a retention time is predicted) is a fixed gradient. In embodiments, the HPLC methods represented in the training data and/or for which a RT is predicted have parameters selected from: a flow rate of 0.2 millilitre per minute, and an elution power of about 1.05, 1.50 and 1.95 (for the mobile phase or components thereof, for example determined as a summarised value for all components of the mobile phase at the respective time point) at 3 time points, such as time points of 0, 15 and 30 minutes.

In an embodiment, the HPLC method parameters for which retention time is predicted at step (b) are constrained to be within respective predetermined ranges and/or selected from respective predetermine sets of values, and/or wherein the training dataset comprises sets of chromatographic data obtained using HPLC method parameters within respective ranges and/or respective sets of values, and the method HPLC method parameters for which retention time is predicted at step (b) are within said ranges and/or selected from said sets of values.

In an embodiment, the elution phase gradient is a non-fixed gradient.

In an embodiment, the machine learning model is an Extreme Gradient Boosted model (e.g. extreme gradient-boosted trees), a Gradient Boosted model (e.g. gradient boosted trees), a Random Forest model, a Lasso regression model, or a Support Vector Machine, preferably an Extreme Gradient Boosted model.

In an embodiment, the machine learning model is trained to minimise differences between predicted retention times and corresponding retention times in training data.

In an embodiment, the machine learning model has been trained using a dataset comprising data for at least 100, at least 200, at least 300, at least 400, at least 500, or at least 600 compounds.

In an embodiment, the dataset comprises at least 2, 3, 4, or 5 sets of chromatographic data comprising a retention time for the respective compound and associated HPLC method parameters.

In embodiments, the training data comprises a plurality of data points (e.g. at least 100, 200, 500, 1000, 1500), each data point comprising molecular/physicochemical properties for a compound, corresponding retention time and HPLC methods parameters. In embodiments, the data comprises multiple data points for the same compound with the same HPLC method, multiple data points for the same compound with different HPLC methods, and multiple compounds with the same HPLC methods. It is advantageous for the training dataset to comprise data for different compounds, such as at least 100, at least 200, at least 300, at least 400, at least 500, or at least 600 compounds. It is also advantageous for the training data to comprise data points for different HPLC methods, such as at least 10, at least 20, at least 50, at least 100, or at least 150, at least 200 different sets of HPLC method parameters (where sets of HPLC method parameters are different if at least one method parameter differs). In embodiments, the training dataset comprises a metric indicative of the width of the peak corresponding to the respective compound (i.e. the peak associated with the retention time for the respective compound).

In an embodiment, the HPLC is Reversed-phase chromatography, Normal-phase chromatography, Size-exclusion chromatography, Ion-exchange chromatography, or Hydrophilic interaction liquid chromatography, optionally wherein the HPLC is Reversed-phase chromatography or Normal-phase chromatography.

In an embodiment, the machine learning model has been trained using a set of predictive features selected from a larger set through a feature selection process, and wherein the larger set of predictive features are each selected from: molecular structural properties or physicochemical properties (e.g. molecular descriptors or molecular fingerprints) and HPLC method parameters.

As the second aspect, the present invention provides a computer-implemented method of identifying a set of HPLC method parameters suitable for separating two or more compounds in a composition comprising said compounds is disclosed, the method comprising:
(a) performing the method of any of embodiments of the first aspect for a plurality of sets of HPLC method parameters;
(b) calculating one or more separation performance metrics using the results of step (a); and
(c) identifying one or more set(s) of HPLC method parameters by applying one or more criteria on said separation performance metrics.

A schematic presentation is provided in FIG. 2B. At step 140, values of one or more sets of HPLC method parameters are obtained. This may comprise obtaining values of one or more HPLC method parameters from optimisation algorithm. At step 150, retention times (and optionally a metric indicative of peak width), are predicted by the method of the first aspect (e.g. as illustrated in FIG. 2A). At step 160, using the results of step 150, one or more separation performance metrics are calculated. At step 170, one or more set(s) of HPLC method parameters are identified by applying one or more criteria on said separation performance metrics. At step 180, the results are made available to output, e.g. by outputting them to a user, e.g. through a user interface, to a computing device or database.

The methods of the present aspect may have any of the features described in relation to any other aspect.

In an embodiment, the separation performance metrics comprise one or more of i) the average retention time difference between adjacent peaks corresponding to predicted retention times for the two or more compounds, ii) the gradient duration, and iii) a predicted metric indicative of peak width, optionally comprising both i) and ii).

In an embodiment, the separation performance metrics comprise i) the average retention time difference between adjacent peaks corresponding to predicted retention times for the two or more compounds, wherein the criteria comprises maximising said average retention time difference; ii) the gradient duration, wherein the criteria comprises minimising said gradient duration; and/or iii) predicted peak widths, wherein the criteria comprises minimising said peak widths.

In an embodiment, the one or more sets of HPLC method parameters in step (a) are generated by an optimisation algorithm, optionally a genetic algorithm.

In an embodiment, step (a) comprises running the genetic algorithm until one or more stopping criteria apply, optionally wherein the stopping criteria are selected from: i) a predetermined number of generations has been reached, and ii) the difference between the separation performance metrics associated with one or more sets of HPLC method parameters of a current iteration and the separation performance metrics associated with one or more sets of HPLC method parameters of a previous iteration is below a threshold.

In an embodiment, the genetic algorithm is initialised with a set of HPLC method parameters selected randomly, wherein the genetic algorithm is initialised with a set of HPLC method parameters selected from a predetermined set or range for each HPLC method parameter, and/or wherein the genetic algorithm is initialised with a set of HPLC method parameters provided by a user.

In an embodiment, one or more sets of HPLC method parameters are selected (e.g. by a genetic algorithm) from respective predetermined sets and/or ranges.

In an embodiment, the method comprises presenting to a user one or more (e.g. 5) sets of HPLC method parameters that satisfy the one or more criteria on said separation performance metrics. Presenting to the user can be e.g. through a user interface, to a computing device or database.

According to a third aspect there is provided a method for providing a tool for predicting a HPLC retention time for one or more compounds, the method comprising: (a) obtaining a training dataset comprising the values of one or more structural and/or physicochemical properties for one or more compounds, and for each compound, one or more sets of chromatographic data comprising a retention time for the respective compound and associated HPLC method parameters; and (b) training a machine learning model to predict a retention time for a compound when subjected to HPLC using the one or more HPLC method parameters, using as input the values of the one or more structural and/or physicochemical properties for the compound, and values of said HPLC method parameters.

The methods of the present aspect may have any of the features described in relation to any other aspect.

In another aspect, a computer program product is disclosed, comprising computer readable instructions which, when executed by one or more processors, cause the one or more processors to carry out the method of any of embodiments of any method described herein.

In another aspect, a non-transitory computer-readable medium is disclosed, having stored thereon computer readable instructions which, when executed by one or more processors, cause the one or more processors to carry out the method of any of embodiments of any method.

In another aspect, a system is disclosed, comprising: at least one processor; and at least one non-transitory computer readable medium containing instructions that, when executed by the at least one

The following is presented by way of example and is not to be construed as a limitation to the scope of the claims.

### EXAMPLES

### Example 1

This work aims to evaluate the use of ML to improve the development process of HPLC methods. To increase efficiency, a recommendation tool is developed that suggests suitable parameters for a given sample mixture. These parameters should serve as a starting point for fine-tuning the exact values of the various HPLC parameters by a trained analyst. The tool makes use of a supervised ML model to predict the RT of the analytes in the sample. The model is trained using the following input data: i) Molecules and their molecular descriptors, ii) HPLC method parameters, iii) HPLC column parameters from the hydrophobic-subtraction model, and iv) Chromatographic data.

The tool uses the RT predictions to evaluate different combinations of method parameters to maximise separation between the analytes. A single-page web application is implemented to provide easy access to a user.

A further aim of this work is to reduce the number of different HPLC columns which would be needed during the HPLC method development process. As different vendors sell columns with similar properties, an unsupervised clustering algorithm is used to group columns with similar properties.

### Methodology

### Technical Implementation

All code was written in Python, version 3.9.7 (Rossum, 2021). Machine learning models were implemented using Scikit-Learn, version 1.0.2 (Pedregosa et al., 2011). The Extreme Gradient Boosting model was implemented using the Scikit-Learn wrapper interface of XGBoost, version 1.5.0 (Chen & Guestrin, 2016). Mol files, chemical descriptors, and fingerprints were generated using RDKit, version 2022.03.1 (“RDKit: Open-source cheminformatics”, n.d.; https://www.rdkit.org). PyGAD, version 2.17.0 was used to implement the genetic algorithm (Fawzy, 2021).

### Data Collection and Pre-Processing

### Chromatographic Data

Chromatographic data were collected from Empower Chromatography Data System, which is a chromatography data software. Chromatographic data were downloaded in the JavaScript Object Notation (JSON) format. As every injection can be processed multiple times by the analyst, multiple results per injection exist. The assumption was made, that in the chromatogram with the latest result_id, the peaks were properly integrated and labelled. From these results, the RT and label of every integrated peak and other relevant information such as the method were extracted and stored in a CSV file for further processing.

After every integrated peak was downloaded, data from both Empower locations were merged. Of the downloaded peaks, many did not have a name that could be linked to an in house molecule identifier (e.g. a Roche number). This can be used to download a molecule file identifying the molecule. To link the abbreviation to a Roche number, information from the Roche internal Small Molecule Impurity Database (SMIDB) and the late-stage method database (Excel file) was used. Information from the SMIDB can be fetched using the SMIDB API. Data from the late-stage method database had to be merged manually before exporting the Excel file in CSV format. Both data sources were merged and matched with the peak names from the Empower dataset.

### HPLC Instrument Methods

The parameter settings describing how to run an HPLC experiment are stored in an instrument method. These parameter settings directly influence the retention time of the analytes in the sample. Therefore, this method's parameters are required as input features for the ML model. Data corresponding to the following parameters were extracted: Method_id (SAM document number), Column, Temperature, Mobile Phase A, Mobile Phase B, Mobile Phase C, Mobile Phase D, pH, Gradient (time points and compositions), and Flow rate.

### Mobile Phase Composition

A variable, elution_power, was introduced. The higher the elution power of the mobile phase composition, the shorter the expected retention time of the analytes. To calculate the “elution power” of a mobile phase, each component and its proportion in a mobile phase were extracted from the SAM documents. Using this information, mol % of each component in the mobile phase was calculated. To get the elution power as shown in Table 1, only the main solvents were taken into account, i.e. water, acetonitrile, methanol, and isopropyl alcohol. Mol % of each solvent was multiplied by its elution strength to give the elution power. See Table 2 for the elution strengths of the solvents. In NP, water has the highest elution strength (i.e. more water=shorter retention times), whereas in RP water has the lowest elution strength (i.e. more water=higher retention times). As only values for NP were available (Trappe, 1940), it was decided to take the inverted NP values as elution strength in RP-LC. The elution power of a mobile phase is the sum of the elution strength of each component (e.g. the elution power of mobile phase A in SAM-0200368 is 0.98+0.04=1.02).

TABLE 1
Example for the calculation of the elution power for each component in SAM-0200368.

| method_id | Eluent | Component | Unit | Quantity | Mol % | elution_strength |
|---|---|---|---|---|---|---|
| SAM-0200368 | mobile_A | Water | Remainder | — | 98.21 | 0.98 |
| SAM-0200368 | mobile_A | ACN | % v/v | 5.00 | 1.78 | 0.04 |
| SAM-0200368 | mobile_A | TFA | % v/v | 0.05 | 0.01 | 0.00 |
| SAM-0200368 | mobile_B | CAN | Remainder | — | 99.96 | 2.00 |
| SAM-0200368 | mobile_B | TFA | % v/v | 0.05 | 0.04 | 0.00 |

### Gradient Information

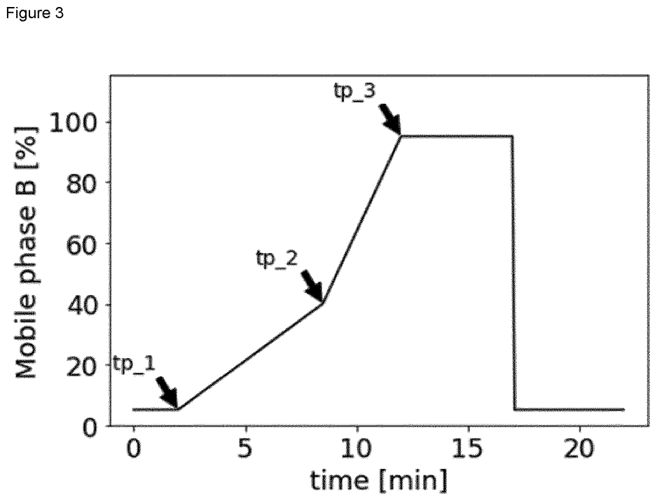

The variables time_point_1, time_point_2, time_point_3 were introduced to describe the mobile phase at three different time points. For each instrument method, these time points were manually extracted together with the mobile phase composition at this time point. FIG. 3 shows the position of the time points for SAM-0200368. At each time point, the elution power was calculated by multiplying the portion of each mobile phase by its elution power and summing up these values. E.g. SAM-0200368: At time point 2, the mobile phase consists of 60% A and 40% B. Therefore, the elution power at this time point is calculated by 0.60*1.02+0.40*2.00=1.41). In Table 3 the time points and elution powers for SAM-0200368 are depicted. These six variables were used as features for the machine learning model to represent the gradient and mobile phases.

TABLE 2
Elution strength of the solvents used for the calculation
of the mobile phase elution power. The inverse values
of the elution strengths in NP-LC were used for RP-LC.

| Solvent | Elution strength NP | Elution strength RP |
|---|---|---|
| Acetonitrile | 0.5 | 2.0 |
| Isopropyl Alcohol | 0.6 | 1.7 |
| Methanol | 0.7 | 1.4 |
| Water | 1.0 | 1.0 |

TABLE 3
Example of a mobile phase composition (tp =
time point, ep = elution power).

| method_id | tp_1 | tp_2 | tp_3 | ep_1 | ep_2 | ep_3 |
|---|---|---|---|---|---|---|
| SAM-0200368 | 2.00 | 8.50 | 12.00 | 1.07 | 1.41 | 1.95 |

### HPLC Column Information

### Accessing the ACDC Database

The Analytical Column Database Compilation (ACDC) contains information on every HPLC and GC column in PTDC. Using the REST API of ACDC, information such as column ID, manufacturer, type of stationary phase, diameters, particle size, product number, USP code, pore size, and surface area were downloaded and stored locally in a CSV file.

### Accessing the USP Database

The parameters of the hydrophobic-subtraction model for more than 750 stationary phases were downloaded from the United States Pharmacopeia (USP) webpage using a web scraper as no REST API was available. Manual download of the data was not an option as the data is constantly updated. Using the developer tools of Google Chrome, the XMLHttpRequest (XHR) sent by the browser while loading the data of the requested webpage were intercepted. These XHR requests were mimicked using a python script. The response sent by the webpage was stored locally in a CSV file.

Combining Analytical Column Information from ACDC Database and PQRI Database

The information fetched from ACDC and the USP webpage were merged to yield a dataset containing stationary phase information for the columns used in the SAM methods including features such as column dimensions, particle sizes, pore sizes, PQRI parameters as well as a number describing how often a certain column was used in a SAM. Fuzzy string matching was used to merge the data sources based on the stationary phase name.

### Chemical Structures

For the prediction of retention times, molecular descriptors and molecular fingerprints were evaluated to represent the chemical structure of a molecule. The Integrated Roche Chemistry Information (IRCI) database is a web application holding the molecular structures of all registered molecules within Roche. Other databases of molecular structures may such be used as e.g. ChEMBL (https://www.ebi.ac.uk/chembl). The IRCI REST API was used to download mol files for every peak in the cleaned Empower dataset (approx. 600 molecules). Using RDKit, the mol files were transformed to a specific RDKit mol file to calculate molecular descriptors using the Mordred package (Moriwaki et al., 2018). Fingerprints were generated using RDKit.

### Combining the Individual Data Sources

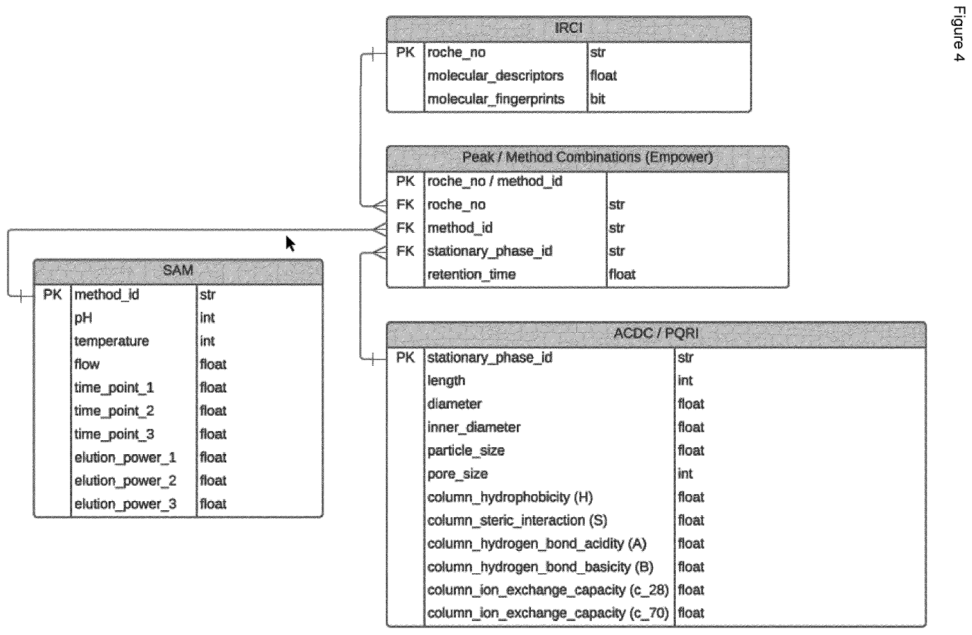

The final step after gathering and pre-processing the individual raw data sources was the consolidation of all data to yield two different datasets ready to be used to train ML models. One dataset holds the molecular descriptors and the other molecular fingerprints. FIG. 4 shows the star schema representing the final datasets.

### Retention Time Prediction

Different ML algorithms were evaluated using the molecular descriptor dataset. The general procedure was a random 75/25 train-test-split before using the GridSearchCV method provided by Scikit-learn for hyper-parameter tuning and performing 10-fold cross-validation on the training set. After defining the ideal values for the hyper-parameters, a new model was trained on the whole training set and its performance was evaluated using the test set. The use of molecular fingerprints for RT prediction was only evaluated using XGB (purely for time constraints; other methods are expected to be suitable).

### Model Evaluation

The performance of the models was evaluated using correlation coefficient (R²), Mean Absolute Error (MAE), and Mean Absolute Percentage Error (MAPE). In addition to R² which is the most common metric for regression problems (Müller & Guido, 2016), MAE was calculated to provide a sense of how good the predictions are in minutes. MAPE was used to account for the magnitude of the RT. R² was used to select the best hyper-parameters during grid search.

Random Train-Test-Split Vs. Unified Train-Test-Split

Before training ML models, the dataset had to be split into a training set and a test set. To perform a random split the train_test_split method from Scikit-learn was used. The results of the models using random train-test-split showed that prediction performance decreased with higher retention time and that the random state defined in the method had an impact on the prediction score. Therefore, a unified train-test-split using an 80/20 split was implemented. For this, the dataset was first sorted by the retention time and then every 5th observation was put into the test set. In addition, it was decided to limit the retention time to 30 min., as there were too few observations with RT>30 min to achieve a train-test-ratio of 5:1.

### Feature Scaling

For linear models and SVM, it is important to scale the features before training the models. If no scaling is applied, features with higher values have a bigger impact than features with smaller values (Müller & Guido, 2016). MinMaxScaler provided by Scikit-learn was used to scale the input features. Usually, each feature is scaled individually, but as the time point and elution power features are in relation to each other (time points 1-3 and elution power 1-3), a special procedure was applied. For both, the time points and the elution powers, a NumPy (Harris et al., 2020) array was created holding all values from the individual time points to fit the scaler. This way, all features have the same min and max values. After fitting the scaler, each feature was then transformed individually. Compared to linear models and SVM, tree-based models do not require scaling of the input data, as each feature is processed individually (Müller & Guido, 2016).

### Dummy Model

A dummy model acts as a baseline to judge the results of other models. If a model achieves a higher score than the dummy regressor, one can say that the predictions are better than predicting random values (Müller & Guido, 2016). A dummy regressor predicts for every observation the same output. In this case, the mean retention time from the observations in the test set (11.6 min) was predicted.

### Feature Selection XGB

As feature importance is calculated in XGB, models with different numbers of features can be evaluated. Decreasing the number of features helps to prevent the model from over-fitting and therefore increases prediction accuracy through better generalization (Müller & Guido, 2016). In addition, a less complex model speeds up both learning and prediction. The following number of features were evaluated: [5, 10, 20, 50, 75, 100, 125, 150, 175, 200, 225, 250, 500, 640]. To select the nth most important features, the “weight” important measure from the XGB model was applied.

### Clustering of Analytical Columns

As different vendors sell columns with similar properties, clustering was evaluated to group columns with similar properties based on the hydrophobic-subtraction model. The goal of this approach was to reduce the variety of columns in the labs. This can be achieved by the optimiser predicting a group of columns (a cluster) in which all columns share similar properties. For the clustering, KMeans and DBSCAN were evaluated. As the ion-exchange capacity (c in the hydrophobic-subtraction model) is pH dependent, two clusterings had to be performed.

### Principal Component Analysis

Before applying a clustering algorithm, Principal Component Analysis (PCA) was evaluated to reduce the number of features from five to two. By rotating the dataset, PCA transforms the dataset to yield uncorrelated features. After rotating the dataset, often only a subset of the features are selected depending on their importance for explaining the data (Müller & Guido, 2016). Using two features allows for plotting the clustering results. All features were scaled using MinMaxScaler before applying PCA.

### KMeans

Using KMeans, the number of clusters has to be defined by the user. This was done iteratively by changing the number of clusters and plotting the results after doing PCA of the dataset. It is important to find the ideal number of clusters. If the number is too high, no reduction in columns will be achieved while a small number leads to clusters containing columns which do not produce similar outputs. Using the defined number of clusters, the clustering was also applied to the dataset without performing PCA beforehand.

### DBSCAN

Compared to KMeans, the number of clusters is defined by the hyper-parameters eps and min_samples. eps defines the maximum distance between data points to be considered as “in the neighbourhood” of each other. min_samples is the number of observations that have to be in the same neighbourhood to form a cluster. If the number of observations in the same neighbourhood is smaller than min_samples, these observations are declared as noise (Müller & Guido, 2016). As every column should be represented by a cluster, min_samples was fixed at 0. The following eps values were tested: [0.04, 0.06, 0.08, 0.1].

### Recommendation Tool

The aim of the recommendation tool is to find suitable HPLC instrument parameters for the separation of a set of predefined molecules. To achieve this, the tool uses the ML model for retention time prediction to find the method parameter combination that maximizes the separation between the analytes. In total, there are 18 method parameters to be defined. These parameters are all features in the ML model: flow rate, temperature, pH, timepoint_1, timepoint_2, timepoint_3, elution_power_1, elution_power_2, elution_power_3, column_length, column_inner_diameter, column_particle_size, column_pore_size, H (hydrophobicity), S (steric interaction), A (hydrogen-bond acidity), B (hydrogen-bond basicity), c (ion-exchange capacity) (https://www.usp.org/resources/pqri-approach-column-equiv-tool). All other features, which are molecular descriptors used by the ML model, are based on the selected analytes.

### Optimisation Methods

Two different optimisation methods were evaluated: Brute Force and Evolutionary Algorithms.

Using brute force, every possible method parameter combination has to be tested to find the one leading to the best separation of the analytes. Using 18 parameters, of which some have more than 40 distinct values, leads to an enormous dataset that cannot be handled by the computer. It was assessed if the possible values for each parameter can be limited to reduce the size of the dataset, but there would have been still millions of combinations to test. Therefore, two evolutionary algorithms were considered: Differential Evolution (DE) and Genetic Algorithm (GA). Evolutionary algorithms are metaheuristics (algorithmic frameworks), often inspired by nature. The goal of a metaheuristic is to solve a complex optimisation problem (Bianchi et al., 2008). In the present work, the optimisation problem is to find 18 method parameters to maximize separation. The strength of metaheuristics is their ability to efficiently explore the search space which is too large for other methods such as brute force (Blum & Roli, 2003). Both, DE and GA, are population-based approaches, meaning multiple candidate solutions are maintained and improved over time by the algorithm (Teghem, 2010). The search space can be limited by defining boundaries using DE and GA. In contrast to DE, GA allows to define the search space using distinct values. As distinct values are a requirement for this work, it was decided to continue the development of the recommendation tool using a GA.

### Genetic Algorithm for Method Parameter Selection

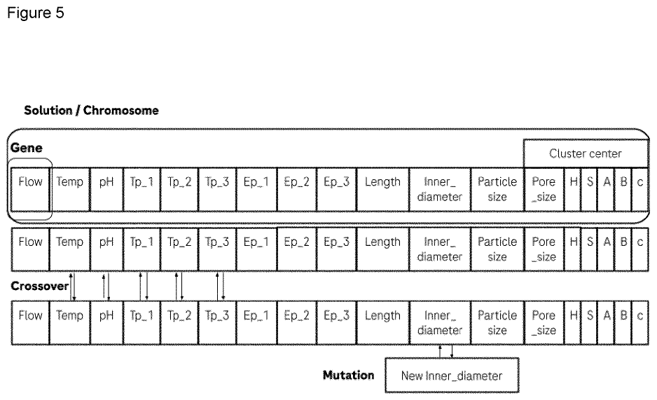

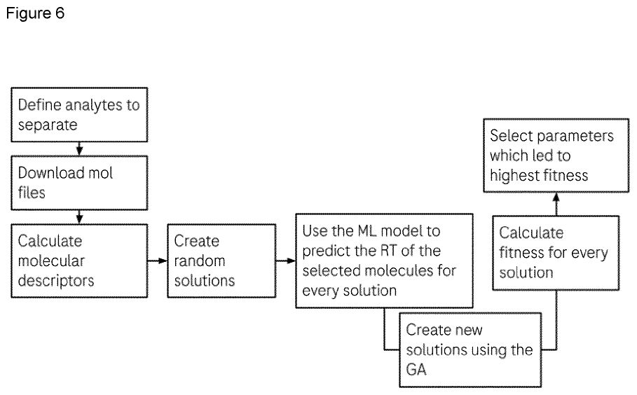

Using PyGAD, a GA can be implemented by providing a fitness function to evaluate candidate solutions, defining the gene space (search space) and setting parameters to guide the optimisation process. Parameters to be defined are, among others, the number of generations, number of parents mating, solutions per generation as well as the mutation rate and crossover behaviour. FIG. 5 shows the important elements of a genetic algorithm. A solution, also called a chromosome, consists of genes. A single gene describes one feature, e.g. flow rate or temperature. Therefore, one solution holds all the information to create an instrument method. In every generation, genes of solutions which reached a high fitness value (parents) are exchanged by crossover to produce the next generations (children). These children are again evaluated using the fitness function. In addition to crossover, a mutation happens at randomly selected genes. During the mutation process, the current value is exchanged for a new value from the gene space of this specific gene.

FIG. 5 shows the general procedure of the optimisation process using GA. The first step in the process is to define molecules to be separated by providing Roche numbers. Using the Roche number, the mol file is downloaded from IRCI and molecular descriptors are calculated for each molecule. Next, the GA produces the first generation of solutions by randomly selecting a value from the gene space for each gene to create a solution. The generated descriptors and the method parameters are merged to give a solution and the retention time for each analyte is predicted. Using the predicted retention times and Equation 1, the fitness value of the current solution is evaluated. The fitness function considers the average retention time difference (Δi) between adjacent peaks (n) and the gradient duration (t). The higher the fitness, the better the expected separation between all analytes. The gradient duration in the fitness function is added as a penalty term to limit run times. Based on the results of the first generation, the GA creates new candidate solutions through crossover between the best solutions of the first generation and random mutation. The process of calculating the fitness for all solutions of the second generation is repeated. If one of the new solutions reaches a higher fitness than the best solution from the first generation, this solution is stored as the best solution. The process is repeated until the pre-defined number of generations is reached. The best_solution is then used to set up the instrument method.

$$fitness = 10 * \frac{1}{n} {\sum }_{i = 1}^{n} Δ i - 0. 5 * t \qquad Equation 1$$

### Implementation of the Genetic Algorithm

The optimizer was set to run for 100 generations using 20 solutions each. To create a new generation, the 10 least fit solutions of the previous generation were replaced by 10 new solutions created through crossover and mutation of the 10 fittest solutions. This behaviour was achieved by using steady-state selection as parent selection type in the parameters (M. Mitchell, 1996). The mutation type was set to random, and the number of genes to mutate was set to one. Even though 18 method parameters had to be selected, the number of genes was set to 14. This is due to allowing only certain combinations of H, S, A, B, and c parameters, namely the cluster centers. If this constraint did not exist, the optimizer could select parameters that lead to non-existing columns. The gene space for the column was set between 0 and 42 which corresponded to the number of unique stationary phases available in the data set. This approach allowed for the evaluation of the optimizer by comparing the predicted retention times and the measured retention times, as every number in the gene space corresponded to exactly one stationary phase. For future use, the tool could also suggest other columns which are in the same cluster as the predicted one. The analyst would then be able to choose a column already available in his or her lab. Based on the selected cluster number, the parameters H, S, A, B, and c were added to the solution. The c value (c_28, c_70) was selected depending on the pH of the solution (e.g. c_28 denotes a pH of less than 7, e.g. 2.8; and c_70 denotes a pH of 7.0; see https://www.usp.org/resources/pqri-approach-column-equiv-tool). It was decided not to apply PCA prior to clustering so that the predicted parameters corresponded to exactly one column. The gene space for the column dimensions was fixed at 150 mm×4.6 mm. The particle size was set to 3.0, which sits between the common 2.7 μm and 3.5 μm particles. The gene space for flow rate was set to [0.75, 1.0, 1.25] to avoid overpressure in the HPLC system in combination with the defined column dimensions. Two options to define the gene space of the gradient (time point and elution power) were defined. The gradient can be fixed. This gradient runs from 5% B to 95% B in 30 min. If the gradient is not fixed, no restrictions are in place and the GA recommends the gradient.

### Optimisation Constraints

Constraints were introduced to avoid the suggestion of impossible values by the optimiser. The following constraints were defined:
time point 1≤time point 2≤time point 3: The run time can only increase over time.
elution power 1≤elution power 2≤elution power 3: The elution power must remain constant or increase during the gradient.
RT latest eluting peak≤time point 3: A peak should elute during the gradient and not in the column wash step.
if time point 2=time point 3 then elution power 2=elution power 3: If the time point does not increase (i.e. only one step in the gradient), the elution power can't be increased as well.

For each violated constraint, the fitness value was multiplied by 0.1.

### Testing the Recommendation Tool

To evaluate the optimiser, two instrument methods and their corresponding Standard Suitability Tests (SST) were randomly selected. For the evaluation, a new XGB model was trained using all data except the Roche number/method_id combinations from the selected instrument methods. Parameters were suggested by the optimiser using both gradient options. When the column was not available with the standard dimensions, the optimiser was re-run with adjusted parameters from an existing column to get the correct retention time predictions. Using an LC-MS (Agilent 1290 LC and Agilent 6150 MSD), the analytes were measured using the optimized parameters. The results were evaluated by comparing predicted vs. measured retention times. In addition, retention times were predicted for all analytes using the parameters stated in the original method.

### Web Application

The web application was implemented using Ploty Dash (“Plotly Technologies Inc. Collaborative data science”, 2015). It allows the analyst to enter multiple analytes by providing information enabling identification of a compound, e.g. the Roche number. In addition, the analyst can choose between the two gradient options. The tool runs the optimisation process multiple times (e.g. five times), as the result of the GA (and other metaheuristics) can vary. This variation is caused by the randomly generated first generation as well as the crossover and mutation behaviour of the algorithm. The trained analyst chooses the run with the highest fitness value overall or based on available columns or predicted RT (e.g. shorter RT).

### Results

### Data Collection and Pre-Processing

### Chromatographic Data

A total of 523′038 injections (1′760′386 peaks) from BS10 and 152′330 injections (424′164 peaks) from BS12 were downloaded (accessed: 08.06.2022). After pre-processing the chromatographic data, a total of 1′122′540 peaks with known SAM document remained. A Roche number could be assigned for 513′877 of these peaks. As the dataset holds the same information multiple times (same molecule and same method_id), the data were grouped by Roche number and method_id and the mean retention time and standard deviation were calculated. This yielded 1′800 unique Roche number/method_id combinations and a total of 600 unique Roche numbers

### HPLC Instrument Methods

240 different SAM documents were processed, which included the manual extraction of the mobile phase composition and the gradient program.

### HPLC Column Information

A total of 1993 unique columns (based on product number) were downloaded from ACDC. These columns were manually linked to the SAM documents. 107 of these 1993 columns were used in one of the SAMs. 757 different column types were available on the USP webpage (accessed: May 2022). The downloaded parameters of the hydrophobic-subtraction model were linked to the columns used in the SAM documents. After the linkage, 94 different columns (based on product number) were left in the dataset, which contained dimensions and hydrophobic subtraction model parameters.

### Chemical Structures

All 1613 available 2D molecular descriptors from the Mordred package were calculated. Every descriptor which could not be calculated for every molecule was later dropped from the dataset. This yielded a dataset with 622 different descriptors. The RDKit was used to calculate morgan fingerprints with 1024 bits for each mol file.

Retention Time Prediction using Molecular Descriptors

### Evaluation of Different Machine Learning Models

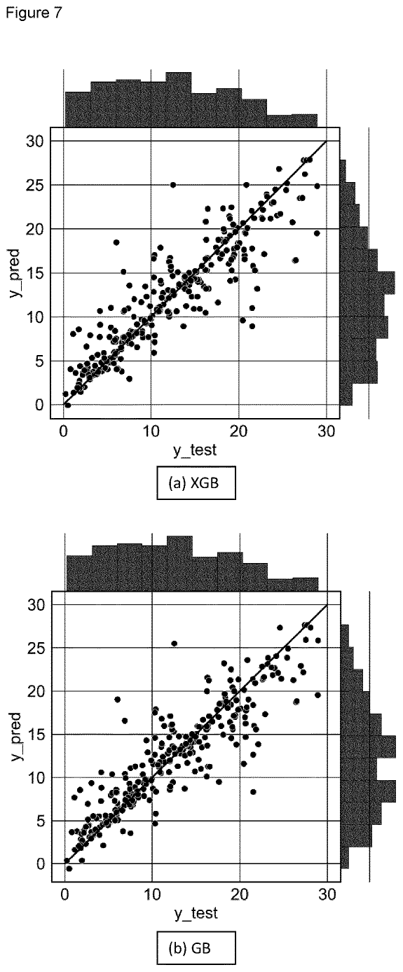

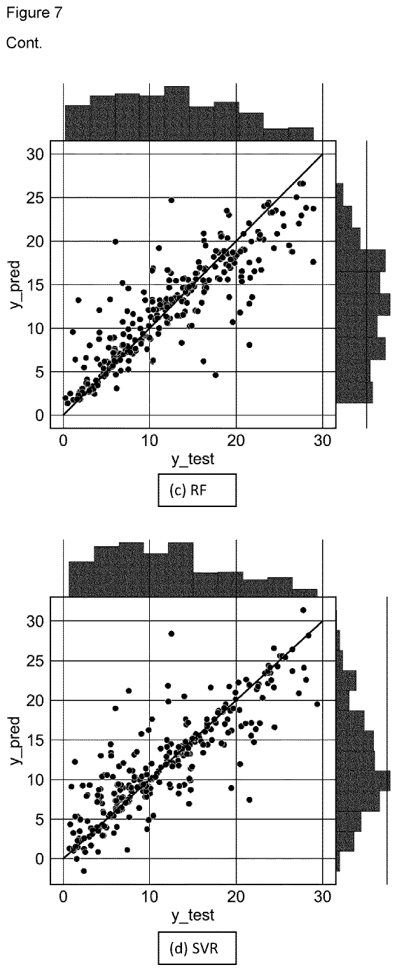

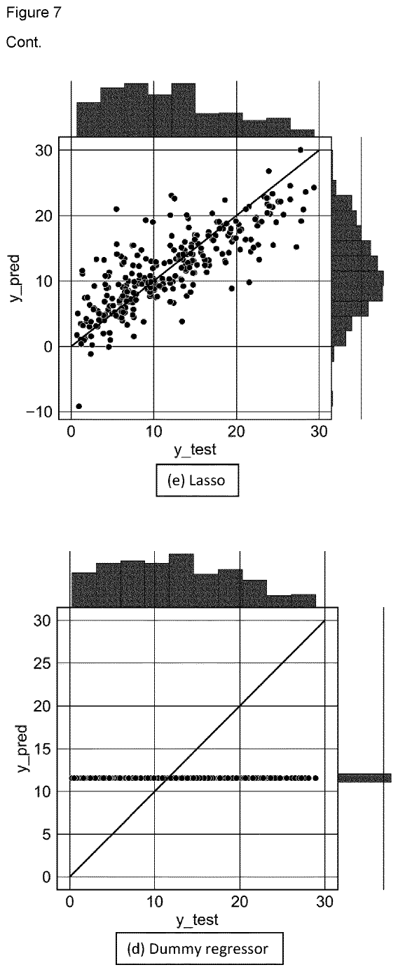

The linear lasso model achieved a prediction score (R²) of 0.663 using the following hyper-parameters: alpha: 0.001, tolerance: 1e-06, max_iterations: 500. SVR achieved an R² of 0.743 and a MAE of 2.4 min using C: 1000 and gamma: 0.01. The tree-based model with the lowest prediction performance was random forest with an R² of 0.770 using bootstrap: True, max_depth: 16, min_samples leaf: 1, num_estimators: 175. GB and XGB showed similar results with an R² of 0.802 and 0.811 respectively. For GB, a learning rate of 0.1 was used together with a max_depth of 5, min_samples leaf of 100 and 1500 estimators. For training the XGB model, a learning rate of 0.05, max_depth of 4, max_leaves of 0, min_child weight of 4 and 750 estimators were used. The dummy regressor, which was used to define a baseline score, achieved an MAE of 5.9 min by always predicting the mean retention time of the training data. This baseline score was outperformed by every evaluated model. The results of the evaluated models are summarized in Table 4 and the prediction error plots are depicted in FIG. 7. While XGB and GB showed a good fit over the entire RT range, RF and Lasso overestimated RT in the lower range and underestimated RT for observations having RT>20 min. SVR achieved similar performance over the entire RT range but achieved lower performance overall compared to XGB and GB. The results show that the two gradient boosting models have the highest prediction performance. As model training is significantly faster using XGB compared to GB, it was decided to further optimise the XGB model.

TABLE 4
Prediction performance of the evaluated ML models prior to feature
selection using random train-test- split and molecular descriptors.

| Model | R² | MAE | MAPE |
|---|---|---|---|
| Dummy | −0.011 | 5.9 | 123.3 |
| Lasso | 0.663 | 2.9 | 47.4 |
| SVR | 0.743 | 2.4 | 37.5 |
| RF | 0.770 | 2.2 | 31.8 |
| GB | 0.802 | 2.0 | 27.3 |
| XGB | 0.811 | 1.9 | 26.5 |

### Tuning XGB Model

The performance of the XGB model was further increased by evaluating the “unified train-test-split” and applying feature selection.

### Unified Train-Test-Split

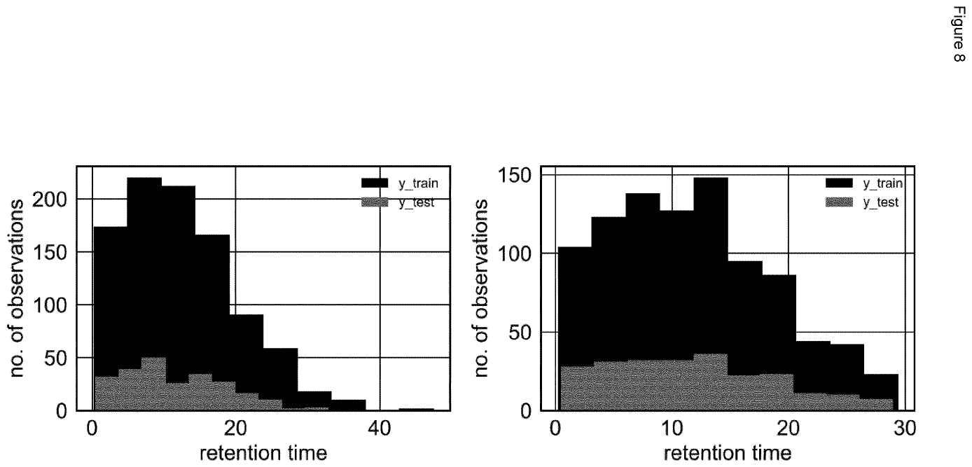

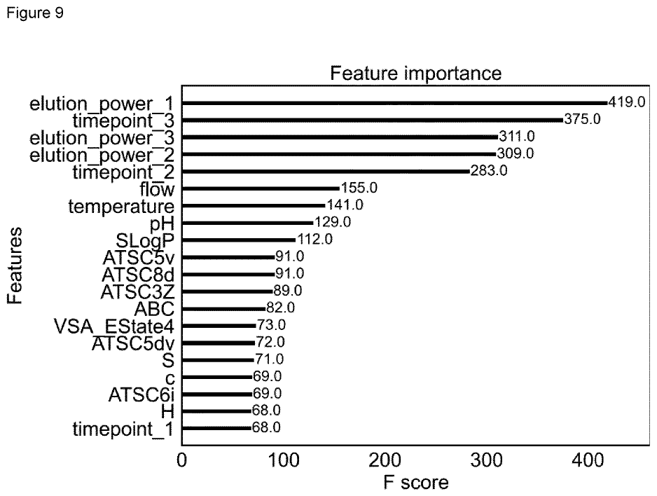

FIG. 8 shows the histograms of both sampling methods before limiting the retention time. Using this sampling approach, R² was increased from 0.811 to 0.830, while MAE was reduced from 1.9 min to 1.6 min. The same hyper-parameter settings were used as described in subsection 3.3.1. FIG. 9 shows the 20 most important features of the XGB model when using unified train-test-split. Apart from the instrument method features, the most important features are:
SLogP: Octanol-water partition coefficient (Wildman & Crippen, 1999).
ATSC: Autocorrelation of a Topological Structure, also known as centered Moreau-Broto autocorrelation. ATSC measures the distribution of atomic properties on a molecular graph (D. R. Todeschini & Consonni, 2020).
ABC: Atom-bond connectivity index that displays an excellent correlation with the heat of formation of alkanes (Estrada, 2008).
VSA_EState: Hybrid of van der Waals surface area and electrotopological state (Guha & Willighagen, 2012).

### Feature Selection

Table 5 shows model performance using different numbers of features. The highest score was achieved using the 65 most important features. As the required features particle_size and inner_diameter were not in the top 65, these two features were added manually. Using feature selection, the number of features was reduced from 640 to 67 while increasing the prediction score. The following hyperparameters were used for the evaluation: gamma: 0, learning_rate: 0.05, max_depth: 4, ‘max_leaves: 0, min_child_weight: 4, n_estimators: 750.

TABLE 5
Prediction performance of the XGB model as a function
of the most important features. The lowest MAE
was reached using 65 features and more.

| No. of features | R² | MAE |
|---|---|---|
| 5 | 0.753 | 2.2 |
| 10 | 0.799 | 1.8 |
| 20 | 0.807 | 1.8 |
| 50 | 0.818 | 1.7 |
| 65 | 0.824 | 1.6 |
| 75 | 0.827 | 1.6 |
| 100 | 0.827 | 1.6 |
| 150 | 0.827 | 1.6 |
| 200 | 0.827 | 1.6 |
| 250 | 0.827 | 1.6 |
| 640 | 0.827 | 1.6 |

Hyper-Parameter Tuning after Feature Selection

After significantly reducing the number of features in the model, the hyper-parameters of the XGB were again evaluated using the parameter grid depicted in Code Listing below.

```
param_grid_feature_selection = {
  ’ n_estimators ’: [1, 10 , 20 , 50 , 100 , 200 , 500 , 750 , 1000 , 1250 , 1500 ],
  ’ learning_rate ’: [0.01 , 0.05 , 0.1],
  ’ max_depth ’: [3, 4, 5],
  ’ max_leaves ’: [0],
  ’ min_child_weight ’:[4],
  ’ gamma ’: [0]}
```

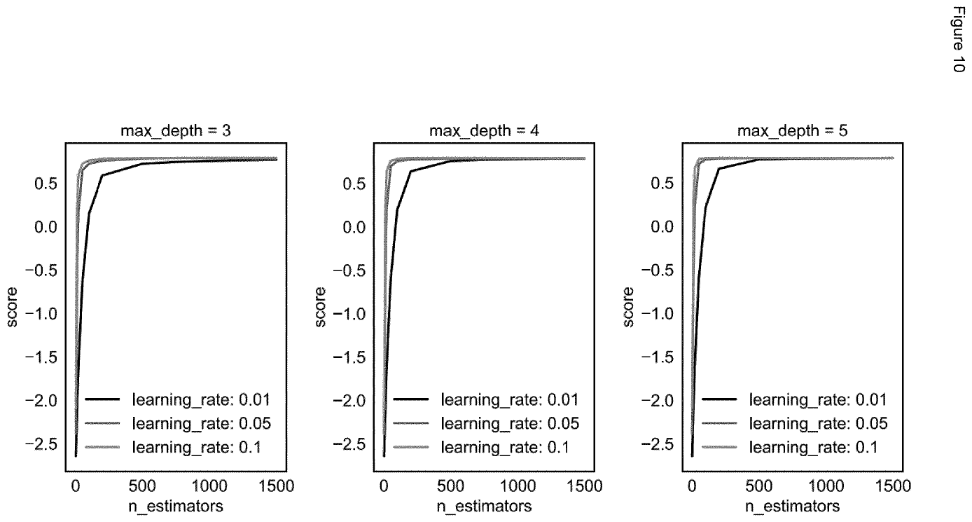

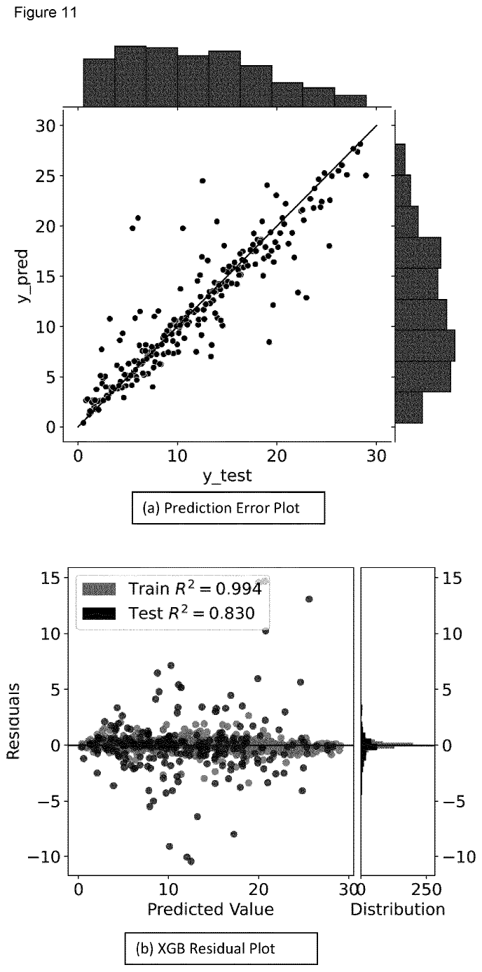

FIG. 10 shows the results with varying n_estimators, max_depth and learning_rate values. Best R² was achieved using the following parameters: n_estimators: 750, learning_rate: 0.05, max_depth: 4, ‘max_leaves: 0, min_child_weight: 4, and gamma: 0. All other parameters used the default value. Through unified train-test-split and feature selection followed by hyper-parameter tuning, R² was increased from 0.811 to 0.827, MAE was decreased from 1.9 min to 1.6 min and MAPE was reduced from 26.5% to 21.5%. In Table 6, the mean test score of the 10-fold CV as a function of the number of estimators is shown. Using more than 750 estimators, a small decrease in prediction score was observed, which is an indication of over-fitting. FIG. 11 shows the prediction error and residuals plot of the final model.

TABLE 6
Mean test score of the XGB model after unified train-test-split
and feature selection using different numbers of estimators.
The highest score was achieved using 750 estimators. A decrease
in performance was observed using 1000 and more estimators.

| n_estimators | 100 | 250 | 500 | 750 | 1000 | 1250 | 1500 | 2000 |
|---|---|---|---|---|---|---|---|---|
| Score | 0.78059 | 0.81042 | 0.81604 | 0.81675 | 0.81670 | 0.81624 | 0.81613 | 0.81579 |

### Retention Time Prediction Using Molecular Fingerprints

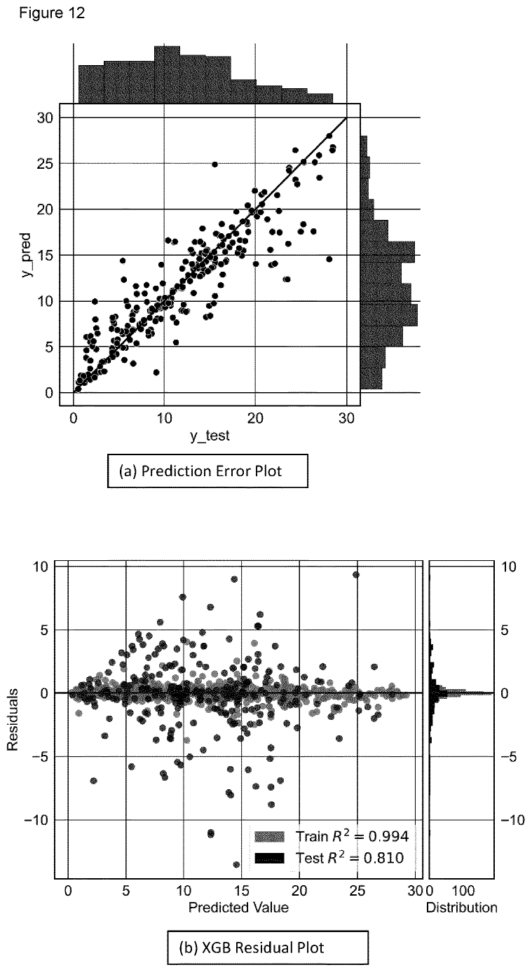

The results using molecular fingerprints and random train-test-split for RT predictions are depicted in FIG. 12. The model achieved a R² of 0.810, a MAE of 1.9 min and a MAPE of 26.4%. These values are comparable to the model using molecular descriptors. The best parameters evaluated using GridSearchCV were a learning_rate of 0.05, 3000 estimators, a max_depth of 4, max_leaves of 0, min_child_weight of 4 and gamma 0.

### Clustering of Analytical Columns

Clustering with PCA

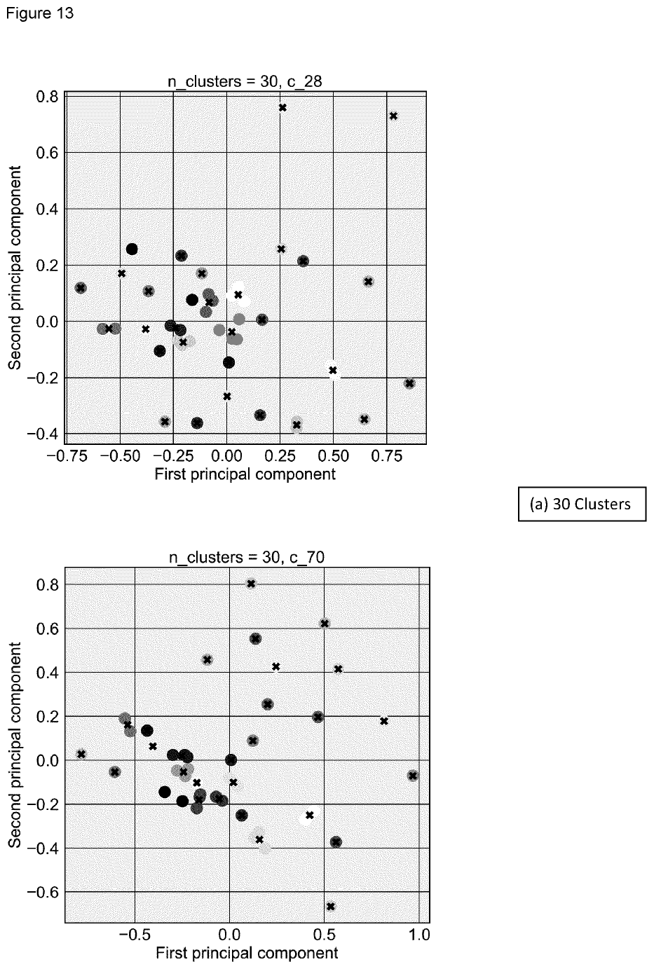

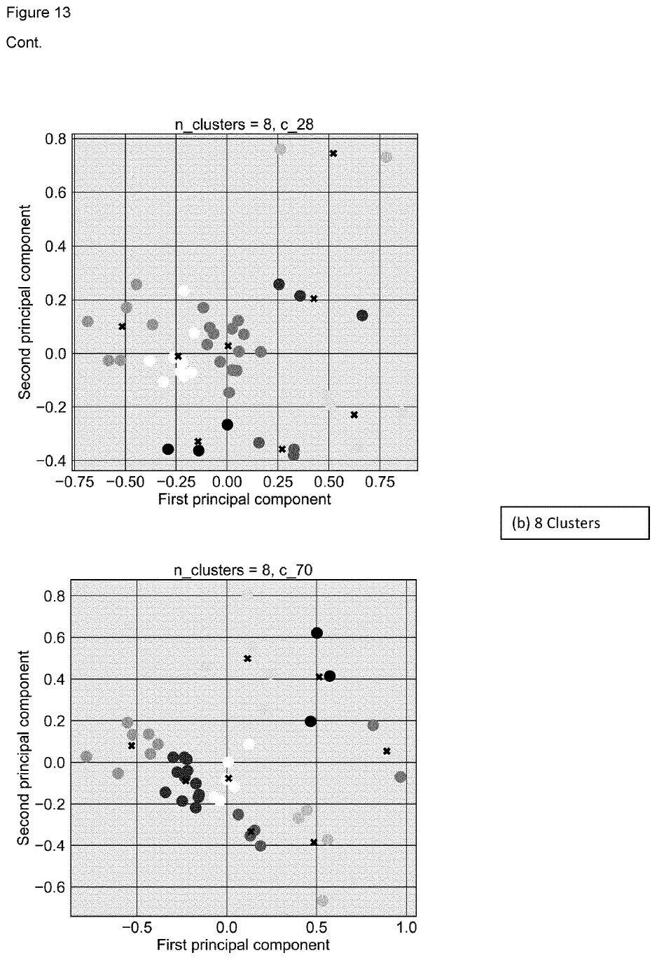

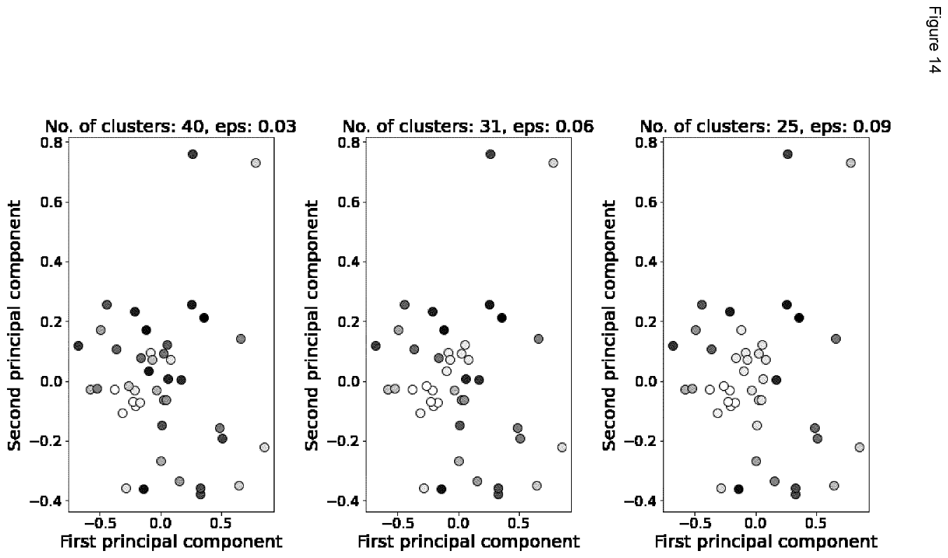

With the reduction from five to two features (first and second principal component), approx. 85% of the variance of the dataset can be explained. FIG. 13 shows the results of the KMeans clustering after PCA was applied. The pH dependence of the ion-exchange parameter “c” is visible by comparing the distribution of the clusters in both plots. As described, min_samples was fixed at 0 as every column should form a cluster when using DBSCAN. FIG. 14 shows the formed cluster using DBSCAN in combination with PCA using different values for eps. It was decided to use the KMeans algorithm for the optimizer as KMeans can also be used to predict the cluster for new observations (Pedregosa et al., 2011).

### Optimiser

It was decided to omit the PCA step and to use 43 instead of 30 clusters to test the optimiser. Using 43 (i.e. the number of different stationary phases in the data set) clusters, the optimiser evaluates the parameters of one specific column. This way, the predicted retention times can be compared to the measured retention times, because the actual column parameters were used for the prediction and not the parameters of the cluster center. For the evaluation of the optimiser, two SAM methods were chosen randomly: SAM-0114188 and SAM-0113392.

### Comparison of Measured and Predicted RT Using SAM Method Parameters

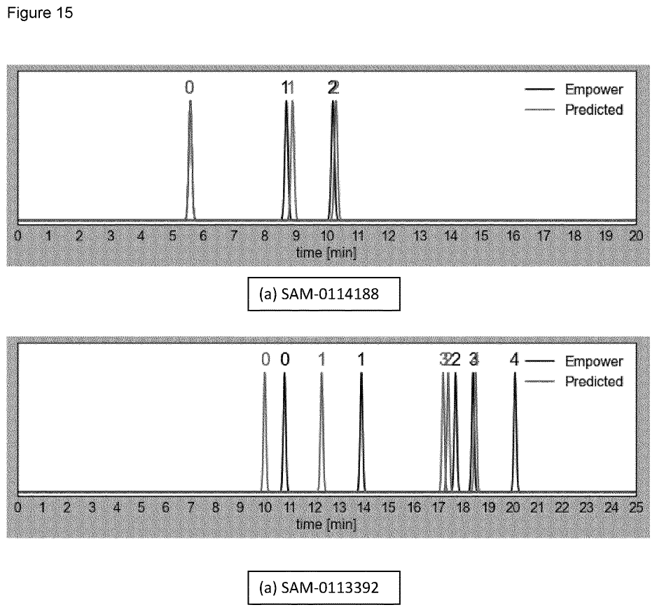

The first test was the prediction of the RT for every molecule described in one of the two SAM documents. The model for the optimiser was trained without a test set but every Roche number/method_id combination from the SAMs was dropped before training the model. In FIG. 15, the measured RT (from the Empower data) for each molecule using the specified method and the predicted RT using the same method parameters are depicted. The overall accuracy of the predictions is good with MAE of 0.10 min (SAM-0114188) and 1.09 min (SAM-0113392). The elution order of the predicted RT corresponded to the measured results for SAM-0114188. A change in elution order was observed for peak no. 2 and peak 3 in SAM-0113392 (FIG. 15b).

### Comparison of Predicted and Measured RT Using Optimiser Output

### SAM-0114188

Table 7 shows the output of the optimizer for the molecules used in SAM-0114188 while not using the fixed gradient. The optimizer ran five times of which each solution had the same fitness. The method parameters differed slightly between each solution but were in the same range (e.g. suggested pH is acidic in every solution). The suggested method parameters of the 5th run (index 4) were evaluated in the laboratory. Cluster 6 corresponded to a Waters Symmetry C18 column. Mobile phase A consisted of water+0.1% TFA and mobile phase B of acetonitrile+0.1% TFA.

TABLE 7
Optimizer output for molecules in SAM-0114188 without gradient
restrictions. In all outputs, flow rate was 0.75, tp1 was
0.0, tp3 was 26.0, ep1 was 1.15, ep2 was 1.25, column length
was 150.0, column diameter was 4.6, particle size was 3.5,
pore size was 120.0, and fitness was 35.062.

| index | temp | pH | Tp2 | Ep3 | cluster |
|---|---|---|---|---|---|
| 0 | 25.0 | 3.0 | 10.0 | 1.30 | 32.0 |
| 1 | 25.0 | 2.0 | 10.0 | 1.30 | 32.0 |
| 2 | 20.0 | 3.0 | 10.0 | 1.35 | 32.0 |
| 3 | 20.0 | 3.0 | 6.0 | 1.30 | 32.0 |
| 4 | 25.0 | 2.0 | 10.0 | 1.35 | 6.0 |

Table 8 shows the output of the optimizer for SAM-0114188 using the fixed gradient. Cluster 6 corresponded to a Waters Symmetry C18 column. Mobile phase A was water+0.1% TFA and mobile phase B acetonitrile+0.1% TFA.

TABLE 8
Optimizer output for molecules in SAM-0114188 using the
fixed gradient only showing the evaluated solution.

| | | | |
|---|---|---|---|
| Index | 4 | Ep2 | 1.50 |
| Flow rate | 1.0 | Ep3 | 1.95 |
| Temperature | 20.0 | Column length | 150.0 |
| pH | 2.0 | Column diameter | 4.6 |
| Tp1 | 0.0 | Particle size | 3.5 |
| Tp2 | 15.0 | Pore size | 120.0 |
| Tp3 | 30.0 | Cluster | 6.0 |
| Ep1 | 1.05 | Fitness | 17.587 |

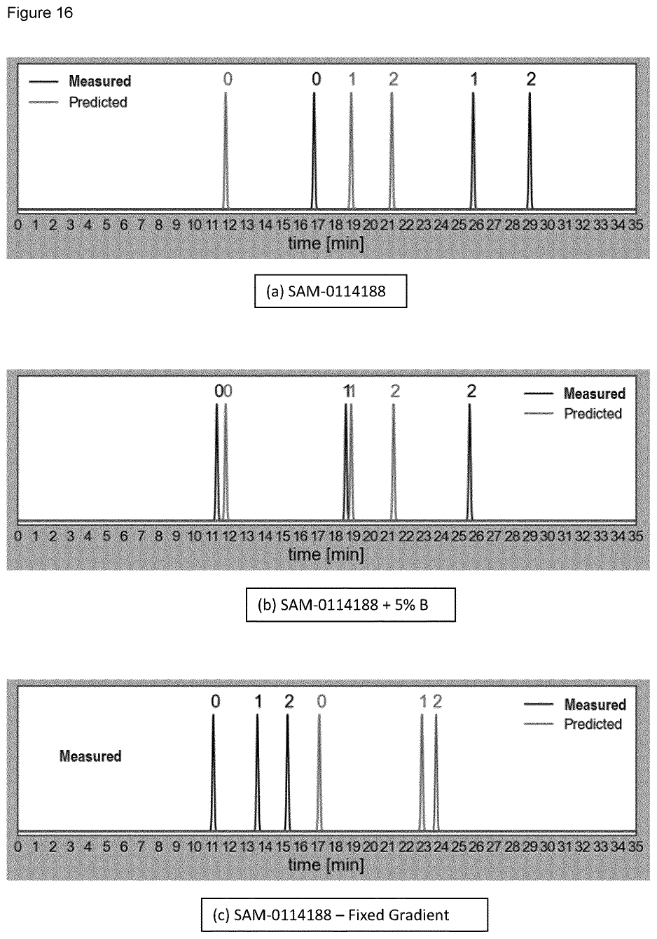

The comparison of the predicted and measured RT are shown in FIG. 16. The suggested method parameters without the fixed gradient (FIG. 16a) lead to good separation between all peaks and the peaks eluted in the correct order. The difference between predicted and measured RT was not within MAE for any peak. Peak 2 eluted while % B reached 95 during the column cleaning step of the gradient. Another test was conducted with a 5% increase of B at every time point of the predicted gradient. The RT difference between predicted and measured decreased for every peak although the RT difference for peak 2 was still over 5 min (see FIG. 16b). Using the “fixed” gradient conditions, the measured peaks showed good separation as well as the correct elution order. In contrast to the results without a fixed gradient, the analytes eluted too early as depicted in FIG. 16c.

### SAM-0113392

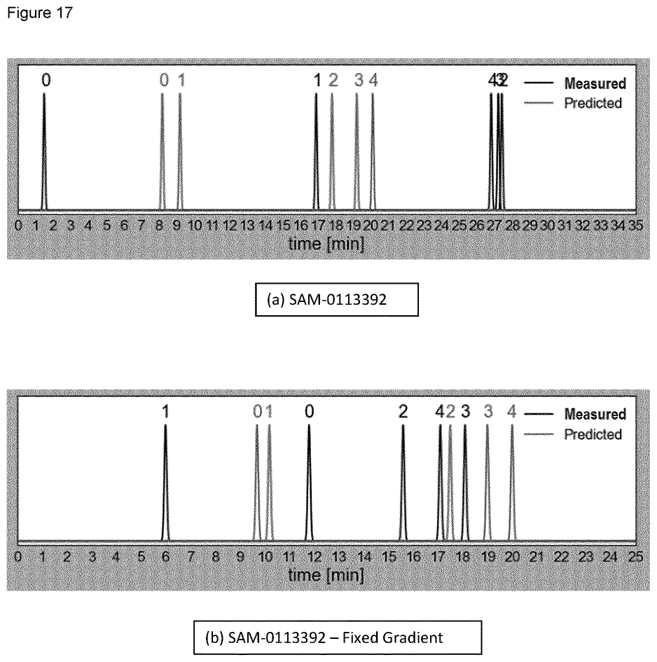

Table 9 shows the optimiser output for SAM-0113392 without using gradient restrictions. The displayed solution is the one tested in the lab. Cluster 1 corresponded to a Phenomenex Kinetex Biphenyl column. The following mobile phases were used to achieve a pH of 3: A: Water+0.01% TFA, B: acetonitrile+0.01% TFA. The comparison between the predicted RT by the optimiser and the measured RT are depicted in FIG. 17a. While Peak 0 eluted too early compared to the predicted RT, peaks 2, 3 and 4 eluted too late without showing good separation among each other.

TABLE 9
Optimiser output for the molecules in SAM-0113392 without gradient
restrictions. Only the evaluated solution is shown.

| | | | |
|---|---|---|---|
| Index | 0 | Ep2 | 1.45 |
| Flow rate | 0.5 | Ep3 | 1.50 |
| Temperature | 40.0 | Column length | 150.0 |
| pH | 3.0 | Column diameter | 3.0 |
| Tp1 | 0.0 | Particle size | 2.6 |
| Tp2 | 6.0 | Pore size | 120.0 |
| Tp3 | 20.0 | Cluster | 1 |
| Ep1 | 1.30 | Fitness | 10.120 |

Table 10 shows the optimiser output for SAM-0113392 using the fixed gradient. The displayed solution is the one tested in the lab. Cluster 11 corresponded to a Waters Acquity UPLC CSH C18. Mobile phase A consisted of 10 mM ammonium acetate in water, adjusted to pH 6 using acetic acid. Mobile phase B consisted of acetonitrile+0.01% acetic acid. The comparison between the predicted RT by the optimiser and the measured RT are shown in FIG. 17b. Peak 1 eluted significantly earlier than predicted, whereas peak 3 and 4 changed the elution order. Compared to the results without gradient restrictions, the measured RT are more accurate and good separation between all peaks was observed.

TABLE 10
Optimizer output for the molecules in SAM-0113392 using
the fixed gradient only showing the evaluated solution.

| | | | |
|---|---|---|---|
| Index | 0 | Ep2 | 1.50 |
| Flow rate | 0.2 | Ep3 | 1.95 |
| Temperature | 45.0 | Column length | 150.0 |
| pH | 6.0 | Column diameter | 2.1 |
| Tp1 | 0.0 | Particle size | 1.7 |
| Tp2 | 15.0 | Pore size | 120.0 |
| Tp3 | 30.0 | Cluster | 11 |
| Ep1 | 1.05 | Fitness | 10.872 |

### Web Application

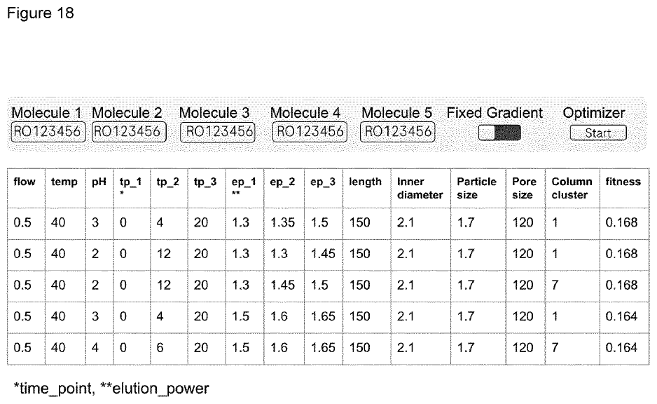

FIG. 18 shows the graphical user interface of the web application prototype. The web app was created using Plotly Dash (“Plotly Technologies Inc. Collaborative data science”, 2015) and allows a user to define up to five molecules described as Roche numbers. The application allows the user to choose between the fixed gradient, where only the flow rate, temperature, pH and column are optimised and the unrestricted option, where every parameter is optimised except the column dimensions. The optimisation process can be started by clicking the start button. Once the optimisation is run, the solutions appear on the results card. From the produced solutions, a trained analyst chooses the solutions with the highest fitness. If the suggested gradient does not seem reasonable, e.g. too flat, the analyst may choose another solution. Often, many solutions will have the same fitness value. In this case, the solution can be chosen also based on available columns.

### CONCLUSION

In summary, a dataset of available HPLC data was used to train an ML model, which in turn was used to optimise analytical method development using a GA. The developed tool can be used to suggest HPLC method parameter settings for a set of pre-defined analytes, which can serve as a starting point for the analytical chemist. To evaluate the different solutions proposed by the GA, an XGB model was used to predict the RTs of the analytes. The XGB model was trained using n=1191 observations which consisted of 49 molecular descriptors and 18 method parameters each. The model achieved R²=0.827 and MAE=1.6 min. To provide access to the tool, a web application prototype was implemented. Based on the predicted RTs using the optimised parameters, the tool was able to find suitable settings for all evaluated cases. To our knowledge, the approach of predicting RT considering method parameters has not yet been developed in other studies. Addition of more data in the training dataset is expected to increase the RT prediction accuracy of the ML model. Once the tool is deployed, it has the potential to increase efficiency by reducing the need for a costly and time-consuming one-factor-at-a-time approach in the development process of new HPLC methods.

### REFERENCES

All references cited herein are incorporated herein by reference in their entirety and for all purposes to the same extent as if each individual publication or patent or patent application was specifically and individually indicated to be incorporated by reference in its entirety. The specific embodiments described herein are offered by way of example, not by way of limitation. Any sub-titles herein are included for convenience only, and are not to be construed as limiting the disclosure in any way.
Bianchi, L., Dorigo, M., Gambardella, L. M., & Gutjahr, W. J. (2008). A survey on metaheuristics for stochastic combinatorial optimization. Natural Computing, 8 (2), 239-287.
Blum, C., & Roli, A. (2003). Metaheuristics in combinatorial optimization: Overview and conceptual comparison. ACM Computing Surveys (CSUR), 35 (3), 268-308.
Bouwmeester, R., Martens, L., & Degroeve, S. (2019). Comprehensive and Empirical Evaluation of Machine Learning Algorithms for Small Molecule LC Retention Time Prediction. Analytical Chemistry, 91 (5), 3694-3703.
Chen, T., & Guestrin, C. (2016). XGBoost: A Scalable Tree Boosting System. Proceedings of the 22nd ACM SIGKDD International Conference on Knowledge Discovery and Data Mining, 785-794.
Dong, M. W. (2006). Modern HPLC for practicing scientists. John Wiley & Sons.
Dong, M. W., & Guillarme, D. (2013). Newer developments in HPLC impacting pharmaceutical analysis: A brief review. American Pharmaceutical Review, 16, 36-43.
Estrada, E. (2008). Atom-bond connectivity and the energetic of branched alkanes. Chemical Physics Letters, 463 (4-6), 422-425.
Gad, A. F. (2021). Pygad: An intuitive genetic algorithm python library. CoRR, abs/2106.06158.
Guha, R., & Willighagen, E. (2012). A Survey of Quantitative Descriptions of Molecular Structure. Current Topics in Medicinal Chemistry, 12 (18), 1946-1956.
Haddad, P. R., Taraji, M., & Szücs, R. (2021). Prediction of Analyte Retention Time in Liquid Chromatography. Analytical Chemistry, 93 (1), 228-256.
Harris, C. R., Millman, K. J., van der Walt, S. J., Gommers, R., Virtanen, P., Cournapeau, D., Wieser, E., Taylor, J., Berg, S., Smith, N. J., Kern, R., Picus, M., Hoyer, S., van Kerkwijk, M. H., Brett, M., Haldane, A., del Río, J. F., Wiebe, M., Peterson, P., . . . . Oliphant, T. E. (2020). Array programming with NumPy. Nature, 585 (7825), 357-362.
Héberger, K. (2007). Quantitative structure-(chromatographic) retention relationships. Journal of Chromatography A, 1158 (1-2), 273-305.
Hewitt, E. F., Lukulay, P., & Galushko, S. (2006). Implementation of a rapid and automated high performance liquid chromatography method development strategy for pharmaceutical drug candidates. Journal of Chromatography A, 1107 (1-2), 79-87.
Kaliszan, R. (1977). Correlation between the retention indices and the connectivity indices of alcohols and methyl esters with complex cyclic structure. Chromatographia, 10 (9), 529-531.
Kaliszan, R., & Foks, H. (1977). The relationship between the RM values and the connectivity indices for pyrazine carbothioamide derivatives. Chromatographia, 10 (7), 346-349.
Kensert, A., Bouwmeester, R., Efthymiadis, K., Broeck, P. V., Desmet, G., & Cabooter, D. (2021). Graph Convolutional Networks for Improved Prediction and Interpretability of Chromatographic Retention Data. Analytical Chemistry, 93 (47), 15633-15641.
Mckinney, W. (2010). Data Structures for Statistical Computing in Python. In S. van der Walt & J. Millman (Eds.), Proceedings of the 9th Python in Science Conference (pp. 56-61).
Mitchell, M. (1996). An introduction to genetic algorithms (Vol. 32). MIT Press. Mitchell, T. M. (1997). Machine Learning. McGraw-Hill.
Molnar, I. (2002). Computerized design of separation strategies by reversed-phase liquid chromatography: development of DryLab software. Journal of Chromatography A, 965 (1-2), 175-194.
Morgan, H. L. (1965). The Generation of a Unique Machine Description for Chemical Structures-A Technique Developed at Chemical Abstracts Service. Journal of Chemical Documentation, 5 (2), 107-113.
Moriwaki, H., Tian, Y.-S., Kawashita, N., & Takagi, T. (2018). Mordred: a molecular descriptor calculator. Journal of Cheminformatics, 10 (1), 4.
Müller, A., & Guido, S. (2016). Introduction to Machine Learning with Python: A Guide for Data Scientists. O'Reilly Media. pandas development team, T. (2020). Pandas-dev/pandas: Pandas (Version 1.4.1). Zenodo.
Pedregosa, F., Varoquaux, G., Gramfort, A., Michel, V., Thirion, B., Grisel, O., Blondel, M., Prettenhofer, P., Weiss, R., Dubourg, V., Vanderplas, J., Passos, A., Cournapeau, D., Brucher, M., Perrot, M., & Duchesnay, E. (2011). Scikit-learn: Machine Learning in Python. Journal of Machine Learning Research, 12, 2825-2830.
Plotly Technologies Inc. Collaborative data science. (2015). https://plot.ly
Probst, D., & Reymond, J.-L. (2018). A probabilistic molecular fingerprint for big data settings. Journal of Cheminformatics, 10 (1), 66.
RDKit: Open-source cheminformatics. (n.d.). https://www.rdkit.org/
Rogers, D., & Hahn, M. (2010). Extended-Connectivity Fingerprints. Journal of Chemical Information and Modeling, 50 (5), 742-754.
Rossum, G. v. (2021). Python. https://docs.python.org/3.9/Snyder, L. R. (2007). A New Look at the Selectivity of RPC Columns. Analytical Chemistry, 79 (9), 3254-3262.
Snyder, L. R., Kirkland, J. J., & Dolan, J. W. (2009). Introduction to Modern Liquid Chromatography (Wiley, Ed.; 3rd).
Snyman, J. A., & Wilke, D. N. (2018). Introduction. In Practical mathematical optimization: Basic optimization theory and gradient-based algorithms (pp. 3-40). Springer International Publishing.
Szucs, R., Brown, R., Brunelli, C., Heaton, J. C., & Hradski, J. (2021). Structure Driven Prediction of Chromatographic Retention Times: Applications to Pharmaceutical Analysis. International Journal of Molecular Sciences, 22 (8), 3848.
Teghem, J. (2010). Metaheuristics. From Design to Implementation, El-Ghazali Talbi. John Wiley & Sons Inc. (2009). XXI+593 pp., Publication 978-0-470-27858-1. European Journal of Operational Research, 205 (2), 486-487.
Todeschini, R., & Consonni, V. (2020a). Handbook of Molecular Descriptors. Methods and Principles in Medicinal Chemistry.
Todeschini, R., & Consonni, V. (2020b). Molecular Descriptors for Chemoinformatics. Methods and Principles in Medicinal Chemistry.
Tome, T., Žigart, N., Časar, Z., & Obreza, A. (2019). Development and Optimization of Liquid Chromatography Analytical Methods by Using AQbD Principles: Overview and Recent Advances. Organic Process Research & Development, 23 (9), 1784-1802.
Trappe, W. (1940). Die Trennung von biologischen Fettstoffen aus ihren natürlichen Gemischen durch Anwendung von Adsorptionssäulen. II. Mitteilung: Abtrennung der phosphor und stickstofffreien Lipoidfraktionen. Biochem. z, 305, 150-154.
Wildman, S. A., & Crippen, G. M. (1999). Prediction of Physicochemical Parameters by Atomic Contributions. Journal of Chemical Information and Computer Sciences, 39 (5), 868-873.
Wilson, N., Nelson, M., Dolan, J., Snyder, L., Wolcott, R., & Carr, P. (2002). Column selectivity in reversed-phase liquid chromatography I. A general quantitative relationship. Journal of Chromatography A, 961 (2), 171-193.

### EMBODIMENTS

In the following, further particular embodiments of the present invention are listed. The specific embodiments described herein are offered by way of example, not by way of limitation. Any sub-titles herein are included for convenience only, and are not to be construed as limiting the disclosure in any way.

1. In an embodiment, a computer-implemented method of predicting a HPLC retention time for one or more compounds is disclosed, the method comprising:
(a) obtaining the values of one or more structural and/or physicochemical properties of said compounds, and one or more HPLC method parameters; and
(b) using a machine learning model to predict a retention time for each of said compounds when subjected to HPLC using the one or more HPLC method parameters, wherein the machine learning model has been trained using a training dataset comprising molecular structural properties and/or physicochemical properties for one or more compounds, and for each compound, one or more sets of chromatographic data comprising a retention time for the respective compound and associated HPLC method parameters.

2. In an embodiment, the method of embodiment 1 is disclosed, wherein the machine learning model further predicts a metric indicative of peak width, and the one or more sets of chromatographic data in the training dataset further comprise a metric indicative of the width of the peak for the respective compound.

3. In an embodiment, the method of embodiment 1 or 2 is disclosed, wherein the one or more compounds are individually selected from: a pharmaceutically active agent, and a degradation product thereof.

4. In an embodiment, the method of any of embodiments 1-3 is disclosed, wherein the HPLC method parameters are selected from a group consisting of: HPLC type, flow rate, temperature, pH, column length, column inner diameter, column particle size, column pore size, column steric interaction, column hydrogen bond acidity, column hydrogen bond basicity, column ion exchange capacity (c_28), column ion exchange capacity (c_70), and one or more metrics defining the elution phase gradient.

5. In an embodiment, the method of any of embodiments 1~4 is disclosed, wherein the HPLC method parameters comprise one or more of the parameters selected from a group consisting of: column length, column inner diameter, column particle size, column pore size, column steric interaction, column hydrogen bond acidity, column hydrogen bond basicity, column ion exchange capacity (c_28), column ion exchange capacity (c_70), and one or more metrics defining the elution phase gradient, and optionally one or more parameters selected from a group consisting of: HPLC type, flow rate, temperature, pH.

6. In an embodiment, the method of any of embodiments 1-5 is disclosed, wherein the HPLC method parameters comprise HPLC type, flow rate, temperature, pH, and one or more of the parameters selected from a group consisting of: column length, column inner diameter, column particle size, column pore size, column steric interaction, column hydrogen bond acidity, column hydrogen bond basicity, column ion exchange capacity (c_28), column ion exchange capacity (c_70), and one or more metrics defining the elution phase gradient.

7. In an embodiment, the method of any of embodiments 1-6 is disclosed, wherein the one or more metrics defining the elution phase gradient comprise:
(i) the elution power of each of a plurality of mobile phases; and/or
(ii) metrics defining the time-dependent change in the proportion of a plurality of mobile phases during elution, optionally wherein the one or more metrics defining the time-dependent change in the proportion of a plurality of mobile phase during elution comprise the value of one or more time points corresponding to changes in the rate of change of the proportion of the mobile phases, and/or the value of the rate of change of the proportion of the mobile phases at one or more time points during the elution, and/or metrics describing a function representing the change in proportion of the mobile phases in one or more of (e.g. each of) a plurality of regions in the gradient; and/or
(iii) the mobile phase elution powers at two or more (e.g. three) different time points, optionally wherein the mobile phase comprises a plurality of mobile phases and the elution power at a time point is obtained as a sum of the elution powers of each of the plurality of mobile phases weighted by the respective proportion of each mobile phase.

8. In an embodiment, the method of any of embodiments 1-7 is disclosed, wherein the one or more physicochemical properties comprise one or more molecular descriptors and/or wherein the one or more molecular structural properties comprise a molecular fingerprint and/or one or more structural molecular descriptor.

9. In an embodiment, the method of embodiment 8 is disclosed, wherein the structural molecular descriptors and/or molecular fingerprints are 2D molecular descriptors or molecular fingerprints.

10. In an embodiment, the method of any of embodiments 1-9 is disclosed, wherein the one or more structural and/or physicochemical properties comprise molecular descriptors selected from a group consisting of SLogP, ATSC5v, ATSC8d, ATSC3Z, ABC, VSA_EState4, ATSC5dv, and ATSC6i.

11. In an embodiment, the method of any of embodiments 6-10 is disclosed, wherein the elution phase gradient is a fixed gradient.

12. In an embodiment, the method of any of embodiments 1 to 11 is disclosed, wherein the HPLC method parameters for which retention time is predicted at step (b) are constrained to be within respective predetermined ranges and/or selected from respective predetermine sets of values, and/or wherein the training dataset comprises sets of chromatographic data obtained using HPLC method parameters within respective ranges and/or respective sets of values, and the method HPLC method parameters for which retention time is predicted at step (b) are within said ranges and/or selected from said sets of values.

13. In an embodiment, the method of any of embodiments 6-10 is disclosed, wherein the elution phase gradient is a non-fixed gradient.

14. In an embodiment, the method of any of embodiments 1-13 is disclosed, wherein the machine learning model is an Extreme Gradient Boosted model (e.g. extreme gradient-boosted trees), a Gradient Boosted model (e.g. gradient boosted trees), a Random Forest model, a Lasso regression model, or a Support Vector Machine, preferably an Extreme Gradient Boosted model.

15. In an embodiment, the method of any of embodiments 1-14 is disclosed, wherein the machine learning model is trained to minimise differences between predicted retention times and corresponding retention times in training data.

16. In an embodiment, the method of any of embodiments 1-15 is disclosed, wherein the machine learning model has been trained using a dataset comprising data for at least 100, at least 200, at least 300, at least 400, at least 500, or at least 600 compounds.

17. In an embodiment, the method of embodiment 16 is disclosed, wherein the dataset comprises at least 2, 3, 4, or 5 sets of chromatographic data comprising a retention time for the respective compound and associated HPLC method parameters.

18. In an embodiment, the method of any of embodiments 1-17 is disclosed, wherein the HPLC is Reversed-phase chromatography, Normal-phase chromatography, Size-exclusion chromatography, lon-exchange chromatography, or Hydrophilic interaction liquid chromatography, optionally wherein the HPLC is Reversed-phase chromatography or Normal-phase chromatography.

19. In an embodiment, the method of any of embodiments 1-18 is disclosed, wherein the machine learning model has been trained using a set of predictive features selected from a larger set through a feature selection process, and wherein the larger set of predictive features are each selected from: molecular structural properties or physicochemical properties (e.g. molecular descriptors or molecular fingerprints) and HPLC method parameters.

20. In an embodiment, a computer-implemented method of identifying a set of HPLC method parameters suitable for separating two or more compounds in a composition comprising said compounds is disclosed, the method comprising:
(a) performing the method of any of embodiments 1-19 for a plurality of sets of HPLC method parameters;
(b) calculating one or more separation performance metrics using the results of step (a); and
(c) identifying one or more set(s) of HPLC method parameters by applying one or more criteria on said separation performance metrics.

21. In an embodiment, the method of embodiment 20 is disclosed, wherein the separation performance metrics comprise one or more of i) the average retention time difference between adjacent peaks corresponding to predicted retention times for the two or more compounds, ii) the gradient duration, and iii) a predicted metric indicative of peak width, optionally comprising both i) and ii).

22. In an embodiment, the method of embodiment 20 or 21 is disclosed, wherein the separation performance metrics comprise i) the average retention time difference between adjacent peaks corresponding to predicted retention times for the two or more compounds, wherein the criteria comprises maximising said average retention time difference; ii) the gradient duration, wherein the criteria comprises minimising said gradient duration; and/or iii) predicted peak widths, wherein the criteria comprises minimising said peak widths.

23. In an embodiment, the method of any of embodiments 20-22 is disclosed, wherein the one or more sets of HPLC method parameters in step (a) are generated by an optimisation algorithm, optionally a genetic algorithm.

24. In an embodiment, the method of embodiment 23 is disclosed, wherein step (a) comprises running the genetic algorithm until one or more stopping criteria apply, optionally wherein the stopping criteria are selected from: i) a predetermined number of generations has been reached, and ii) the difference between the separation performance metrics associated with one or more sets of HPLC method parameters of a current iteration and the separation performance metrics associated with one or more sets of HPLC method parameters of a previous iteration is below a threshold.

25. In an embodiment, the method of embodiment 23 or 24 is disclosed, wherein the genetic algorithm is initialised with a set of HPLC method parameters selected randomly, wherein the genetic algorithm is initialised with a set of HPLC method parameters selected from a predetermined set or range for each HPLC method parameter, and/or wherein the genetic algorithm is initialised with a set of HPLC method parameters provided by a user.

26. In an embodiment, the method of any of embodiments 1-25 is disclosed, wherein one or more sets of HPLC method parameters are selected (e.g. by a genetic algorithm) from respective predetermined sets and/or ranges.

27. In an embodiment, the method of any of embodiments 20-26 is disclosed, wherein the method comprises presenting to a user one or more (e.g. 5) sets of HPLC method parameters that satisfy the one or more criteria on said separation performance metrics.

28. In an embodiment, a computer program product is disclosed, comprising computer readable instructions which, when executed by one or more processors, cause the one or more processors to carry out the method of any of embodiments 1-27.

29. In an embodiment, a non-transitory computer-readable medium is disclosed, having stored thereon computer readable instructions which, when executed by one or more processors, cause the one or more processors to carry out the method of any of embodiments 1-27.

30. In an embodiment, a system is disclosed, comprising:
at least one processor; and
at least one non-transitory computer readable medium containing instructions that, when executed by the at least one processor, cause the at least one processor to perform the method of any of embodiments 1 to 27.

1. A computer-implemented method of predicting a HPLC retention time for one or more compounds, the method comprising:
(a) obtaining the values of one or more structural and/or physicochemical properties of said compounds, and one or more HPLC method parameters; and
(b) using a machine learning model to predict a retention time for each of said compounds when subjected to HPLC using the one or more HPLC method parameters,
wherein the machine learning model has been trained using a training dataset comprising molecular structural properties and/or physicochemical properties for one or more compounds, and for each compound, one or more sets of chromatographic data comprising a retention time for the respective compound and associated HPLC method parameters.

2. The method of claim 1, wherein the machine learning model further predicts a metric indicative of peak width, and the one or more sets of chromatographic data in the training dataset further comprise a metric indicative of the width of the peak for the respective compound.

3. The method of claim 1 or 2, wherein the HPLC method parameters are selected from a group consisting of: HPLC type, flow rate, temperature, pH, column length, column inner diameter, column particle size, column pore size, column steric interaction, column hydrogen bond acidity, column hydrogen bond basicity, column ion exchange capacity (c_28), column ion exchange capacity (c_70), and one or more metrics defining the elution phase gradient, e.g, wherein the HPLC method parameters comprise one or more of the parameters selected from a group consisting of: column length, column inner diameter, column particle size, column pore size, column steric interaction, column hydrogen bond acidity, column hydrogen bond basicity, column ion exchange capacity (c_28), column ion exchange capacity (c_70), and one or more metrics defining the elution phase gradient.

4. The method of any of claims 1-3, wherein the one or more physicochemical properties comprise one or more molecular descriptors and/or wherein the one or more molecular structural properties comprise a molecular fingerprint and/or one or more structural molecular descriptor, optionally wherein the one or more structural and/or physicochemical properties comprise molecular descriptors selected from a group consisting of SLogP, ATSC5v, ATSC8d, ATSC3Z, ABC, VSA_EState4, ATSC5dv, and ATSC6i.

5. The method of any of claims 1-4, wherein the HPLC is Reversed-phase chromatography, Normal-phase chromatography, Size-exclusion chromatography, Ion-exchange chromatography, or Hydrophilic interaction liquid chromatography, optionally wherein the HPLC is Reversed-phase chromatography or Normal-phase chromatography.

6. The method of any of claims 1-5, wherein the machine learning model has been trained using a set of predictive features selected from a larger set through a feature selection process, and wherein the larger set of predictive features are each selected from: molecular structural properties or physicochemical properties (e.g. molecular descriptors or molecular fingerprints) and HPLC method parameters.

7. A computer-implemented method of identifying a set of HPLC method parameters suitable for separating two or more compounds in a composition comprising said compounds, the method comprising:
(a) performing the method of any of claims 1-6 for a plurality of sets of HPLC method parameters;
(b) calculating one or more separation performance metrics using the results of step (a); and
(c) identifying one or more set(s) of HPLC method parameters by applying one or more criteria on said separation performance metrics.

8. The method of claim 7, wherein the separation performance metrics comprise one or more of i) the average retention time difference between adjacent peaks corresponding to predicted retention times for the two or more compounds, ii) the gradient duration, and iii) a predicted metric indicative of peak width, optionally comprising both i) and ii).

9. The method of claim 7 or 8, wherein the separation performance metrics comprise i) the average retention time difference between adjacent peaks corresponding to predicted retention times for the two or more compounds, wherein the criteria comprises maximising said average retention time difference; ii) the gradient duration, wherein the criteria comprises minimising said gradient duration; and/or iii) predicted peak widths, wherein the criteria comprises minimising said peak widths.

10. The method of any of claims 7-9, wherein the one or more sets of HPLC method parameters in step (a) are generated by an optimisation algorithm, optionally a genetic algorithm.

11. The method of claim 10, wherein step (a) comprises running the genetic algorithm until one or more stopping criteria apply, optionally wherein the stopping criteria are selected from: i) a predetermined number of generations has been reached, and ii) the difference between the separation performance metrics associated with one or more sets of HPLC method parameters of a current iteration and the separation performance metrics associated with one or more sets of HPLC method parameters of a previous iteration is below a threshold.

12. The method of any of claims 7-11, wherein the method comprises presenting to a user one or more (e.g. 5) sets of HPLC method parameters that satisfy the one or more criteria on said separation performance metrics.

13. A computer program product comprising computer readable instructions which, when executed by one or more processors, cause the one or more processors to carry out the method of any of claims 1-12.

14. A non-transitory computer-readable medium having stored thereon computer readable instructions which, when executed by one or more processors, cause the one or more processors to carry out the method of any of claims 1-12.

15. A system comprising:
at least one processor; and
at least one non-transitory computer readable medium containing instructions that, when executed by the at least one processor, cause the at least one processor to perform the method of any of claims 1 to 14.
